(19) United States
(12) Patent Application Publication
(10) Pub. No.: US 2020/0179415 A1
(43) Pub. Date: Jun. 11, 2020

(54) HIGHLY ACTIVE DRUG COMBINATION FOR TREATMENT OF HEPATITIS C VIRUS

(71) Applicant: Atea Pharmaceuticals, Inc., Boston, MA (US)

(72) Inventors: Jean-Pierre Sommadossi, Boston, MA (US); Adel Moussa, Burlington, MA (US); Kieth M. Pietropaolo, Boxford, MA (US); Xia-Jian Zhou, Arlington, MA (US)

(73) Assignee: Atea Pharmaceuticals, Inc., Boston, MA (US)

(21) Appl. No.: 16/703,599

(22) Filed: Dec. 4, 2019

Related U.S. Application Data
(60) Provisional application No. 62/775,711, filed on Dec. 5, 2018.
(60) Provisional application No. 62/909,486, filed on Oct. 2, 2019.

(51) Int. Cl. A61K 31/685; A61K 31/4178; A61K 9/00; A61P 31/14; C07D 405/14

(52) U.S. Cl. CPC A61K 31/685; A61K 31/4178; C07B 2200/13; A61P 31/14; C07D 405/14; A61K 9/0053

(57) ABSTRACT

A combination is provided of Compound 1 or a pharmaceutically acceptable salt thereof (such as Compound 1-A) and Compound 2 or a pharmaceutically acceptable salt thereof (such as Compound 2-A)

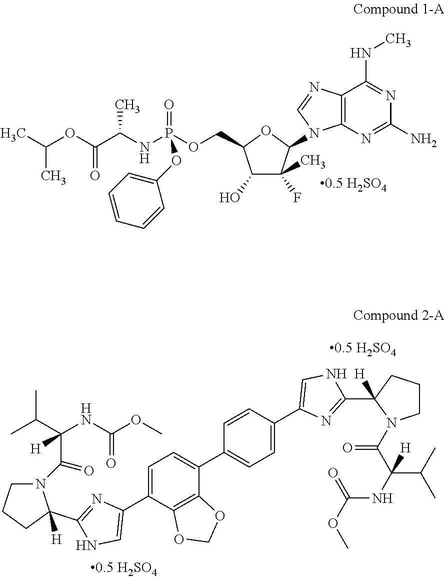 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F ; COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

to treat a host infected with hepatitis C, as well as pharmaceutical compositions and dosage forms, including solid dosage forms thereof.

### CROSS-REFERENCE TO RELATED APPLICATIONS

This application claims the benefit of U.S. Provisional Application No. 62/775,711, filed Dec. 5, 2018, and U.S. Provisional Application No. 62/909,486, filed Oct. 2, 2019. These applications are incorporated by reference in their entireties.

### FIELD OF THE INVENTION

The present invention is a highly active combination of a NS5B polymerase inhibitor and a NS5A inhibitor for anti-hepatitis C therapy, as well as a new solid salt form of the NS5A inhibitor that is advantageous for use in a solid pharmaceutical dosage form.

### BACKGROUND OF THE INVENTION

Hepatitis C (HCV) is an RNA single-stranded virus and member of the Hepacivirus genus. It is estimated that 55-85% of all cases of liver disease are caused by HCV. HCV infection can lead to cirrhosis and liver cancer, and if left to progress, liver failure that may require a liver transplant. Approximately 71 million people worldwide are living with chronic HCV infections and approximately 350,000-500,000 people die each year from HCV-related complications, mostly from cirrhosis and hepatocellular carcinoma.

RNA polymerase is a key target for drug development against RNA single-stranded viruses. The HCV non-structural protein NS5B RNA-dependent RNA polymerase is a key enzyme responsible for initiating and catalyzing viral RNA synthesis. There are two major subclasses of NS5B inhibitors: nucleoside analogs and non-nucleoside inhibitors (NNIs). Nucleoside analogs are anabolized to active triphosphates that act as alternative substrates for the polymerase. Non-nucleoside inhibitors (NNIs) bind to allosteric regions on the protein. Nucleoside or nucleotide inhibitors mimic natural polymerase substrates and act as chain terminators. They inhibit the initiation of RNA transcription and elongation of a nascent RNA chain.

In addition to targeting RNA polymerase, other RNA viral proteins may also be targeted. For example, HCV proteins that are additional targets for therapeutic approaches include NS3/4A (a serine protease) and NS5A (a non-structural mitochondrial protein that is an essential component of HCV replicase with no enzymatic ability that exerts a range of effects on cellular pathways and is required for HCV functionality).

In December 2013, the first nucleoside NS5B polymerase inhibitor sofosbuvir (Sovaldi®, Gilead Sciences) was approved. Sovaldi® is a uridine phosphoramidate prodrug that is taken up by hepatocytes and undergoes intracellular activation to afford the active metabolite, 2′-deoxy-2′-α-fluoro-β-C-methyluridine-5′-triphosphate. Sovaldi® is the first drug that demonstrated safety and efficacy to treat certain types of HCV infection without the need for co-administration of interferon. Sovaldi® is the third drug with breakthrough therapy designation to receive FDA approval.

A number of additional fixed-dose drug combinations have been approved for the treatment of HCV. In 2014, the U.S. FDA approved Harvoni® (ledispasvir, a NS5A inhibitor, and sofosbuvir) to treat chronic hepatitis C virus Genotype 1 infection. Harvoni® is the first combination pill approved to treat chronic HCV Genotype 1 infection. It is also the first approved regimen that does not require administration with interferon or ribavirin. In addition, the FDA approved simeprevir (Olysio™) in combination with sofosbuvir (Sovaldi®) as a once-daily, all oral, interferon and ribavirin-free treatment for adults with Genotype 1 HCV infection.

The U.S. FDA also approved AbbVie's VIEKIRA Pak™ in 2014, a multi-pill pack containing dasabuvir (a non-nucleoside NS5B polymerase inhibitor), ombitasvir (a NS5A inhibitor), paritaprevir (a NS3/4A inhibitor), and ritonavir. The VIEKIRA Pak™ can be used with or without the ribavirin to treat Genotype 1 HCV infected patients including patients with compensated cirrhosis. VIEKIRA Pak™ does not require interferon co-therapy.

In July 2015, the U.S. FDA approved Technivie™ (Ombitasvir/paritaprevir/ritonavir) and Daklinza™ for the treatment of HCV genotype 4 and HCV Genotype 3, respectively. Technivie™ was approved for use in combination with ribavirin for the treatment ofHCV genotype 4 in patients without scarring and cirrhosis and is the first option for HCV-4 infected patients that do not require co-administration with interferon. Daklinza™ was approved for use with Sovaldi® to treat HCV genotype 3 infections. Daklinza™ is the first drug that has demonstrated safety and efficacy in treating HCV Genotype 3 without the need for co-administration of interferon or ribavirin.

In October 2015, the U.S. FDA warned that HCV treatments Viekira Pak and Technivie™ can cause serious liver injury primarily in patients with underlying advanced liver disease and required that additional information about safety be added to the label.

Other current approved therapies for HCV include interferon alpha-2b or pegylated interferon alpha-2b (Pegintron®), which can be administered with ribavirin (Rebetol®), NS3/4A telaprevir (Incivek®, Vertex and Johnson & Johnson), boceprevir (Victrelis™, Merck), simeprevir (Olysio™, Johnson & Johnson), paritaprevir (AbbVie), Ombitasvir (AbbVie), the NNI Dasabuvir (ABT-333), Merck's Zepatier™ (a single-tablet combination of the two drugs grazoprevir and elbasvir), Gilead's Epclusa (sofosbuvir, velpatasvir), Mavyret (glecaprevir and pibrentasvir) manufactured by AbbieVie, and Vosevi (sofosbuvir, velpatasvir, and voxilaprevir).

There remains a strong medical need to develop anti-HCV therapies that are effective and not unduly toxic. The need is accentuated by potential drug resistance. The HCV RNA polymerase exhibits a high rate of replication that contributes to the production of potentially resistant single and double point mutations throughout the genome and the maintenance of viral quasispecies. Resistance mutations have been identified both in vitro and in vivo upon treatment with nearly all monotherapies.

It is therefore an object of the present invention to provide compounds, pharmaceutical compositions, methods, and dosage forms to treat and/or prevent infections of the hepatitis C virus, or disorders associated with a hepatitis C viral infection.

### SUMMARY OF THE INVENTION

The present invention provides a highly active combination of Compound 1, which is a NS5B polymerase inhibitor, or a pharmaceutically acceptable salt thereof, and Compound 2, which is a NS5A inhibitor, or a pharmaceutically acceptable salt thereof, for the advantageous treatment of a hepatitis C infection in a host, typically a human. This combination of two highly active anti-HCV agents acting together with distinct mechanisms can be provided in a desired combined pharmaceutical formulation, such as a solid dosage form, or can be administered separately in a manner that the host receives the benefit of both active agents acting in a concerted biological manner, for example, in a manner that achieves an overlapping pharmacokinetic, plasma and/or AUC.

As established in Example 6 and FIGS. 4A, 4B and 4C, it has been discovered that Compound 1 and Compound 2 show synergistic activity against the hepatitis C virus. It cannot be predicted in advance how two active drugs will interact when administered to a human in a combination regimen. The two drugs can be antagonistic, additive, or synergistic. The combination of the present invention thus unexpectedly acts synergistically to provide an optimal anti-HCV therapeutic effect.

In one nonlimiting embodiment, Compound 1 is provided as a hemisulfate salt. In one non-limiting embodiment, Compound 2 is provided as a di-hemisulfate salt.

Compound 1 is isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate:

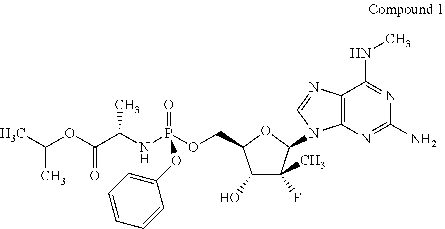 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

Compound 1 was previously described in U.S. Pat. Nos. 9,828,410; 10,000,523; 10,005,811; and, 10,239,911 and PCT Applications WO 2016/21276 and WO 2019/200005 assigned to Atea Pharmaceuticals.

The hemisulfate salt of Compound 1 is shown below as Compound 1-A:

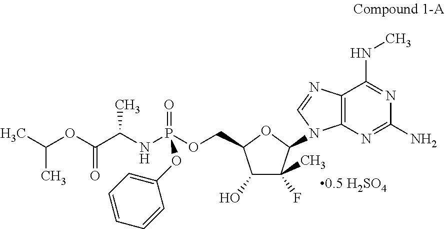 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

Compound 1-A is disclosed in US 2018-0215776 and PCT Applications WO 2018/144640 and WO 2019/200005 assigned to Atea Pharmaceuticals.

Compound 2 is Coblopasvir (or KW-136, methyl N-[(2S)-1-[(2S)-2-[5-[4-[7-[2-[(2S)-1-[(2 S)-2-(methoxycarbonylamino)-3-methylbutanoyl]pyrrolidin-2-yl]-1H-imidazol-5-yl]-1,3-benzodioxol-4-yl]phenyl]-1H-imidazol-2-yl]pyrrolidin-1-yl]-3-methyl-1-oxobutan-2-yl]carbamate):

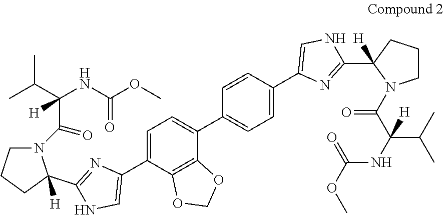 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

Compound 2 is disclosed in WO 2011/075607 and U.S. Patent Application US 2011/0152246 (page 104) assigned to InterMune, Inc.

In one embodiment, Compound 2 is a di-hemisulfate salt, Compound 2-A:

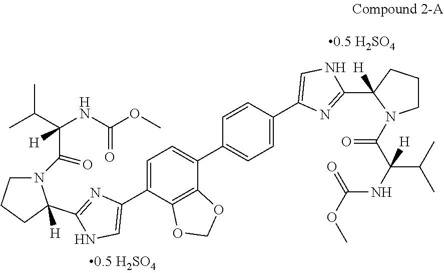 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

The di-hemisulfate salt of Compound 2 has not been disclosed to date. In fact, Coblopasvir has been administered to date as the di-HCl salt. The morphic form of the di-HCl salt of Coblopasvir was disclosed in Chinese Patent Applications No. CN 108904496 and CN 108675998 assigned to Beijing Kawin Technology Share-Holding Co. The assignee described that a crystalline form of the di-HCl salt of Compound 2 is difficult to produce. Despite using large amounts of solvent, different solvent combinations, and a variety of different crystallization techniques, only one crystalline form (Form H) was obtained by Beijing Kawin Technology Share-Holding Co. in Chinese Patent Applications Nos. CN 108904496 and CN 108675998. The Form H was obtained by dissolving Compound 2 in a large amount of MeOH (2-4× the amount by weight of Compound 2), adding HCl, and refluxing.

In contrast, it has now been surprisingly discovered that Compound 2-A can be provided as a well-behaved stable, solid di-hemisulfate salt. As discussed in Example 3 of the present invention, the crystallization of Compound 2 was studied with sixteen different inorganic and organic acids. Each of the acids was tested in at least two different solvents for a total of 48 studies.

From these conditions, it was surprisingly discovered that when the di-hemisulfate acid was used in combination with MeOH as a solvent, a stable, crystalline compound was obtained that is suitable for drug formulation.

The present invention thus provides for the first time an advantageous isolated morphic form of Compound 2-A. The XRPD pattern of the isolated morphic form of the di-hemisulfate salt of Compound 2 is provided in FIG. 1A. In one embodiment, the morphic form of Compound 2-A is characterized by an XRPD pattern comprising at least five, six, seven, eight, nine, or ten 20 values selected from Table 2. In one embodiment, the morphic form of Compound 2-A is characterized by an XRPD pattern comprising 20 values including at least or selected from 7.3+/−0.2°2θ, 7.9+/−0.2°2θ, 12.0+/−0.2°2θ, 12.2+/−0.2°2θ, 14.7+/−0.2°2θ, 15.8+/−0.2°2θ, 16.1+/−0.2°2θ, 16.5+/−0.2°2θ, 18.2+/−0.2°2θ, and 22.7+/−0.2°2θ. In an alternative embodiment, the standard deviation is +/−0.3°2θ+/−0.4°2θ.

It is unusual to provide a stable solid combination dosage form of two compounds that are both in the salt form. Co-formulated combination drugs tend to comprise no pharmaceutically acceptable salts (Epclusa, Vosevi, Zepatier, and Harvoni) or one pharmaceutically acceptable salt (Daklinza). The use of different salt forms in co-formulations may be considered to increase the risk of hygroscopicity, instability of formulation, or otherwise decrease the ease of stable co-formulation which can affect administration or efficacy. Using different salt forms in co-formulations can also be problematic in terms of chemical analysis and meeting regulatory requirements. Compound 1-A and Compound 2-A can be formulated together, perhaps in part because of the high stability and purity of the morphic form Compound 2-A. As described in Example 5, the purity of Compound 2-A was not changed when subjected to 25° C. and 60% RH or 40° C. and 75% RH. Thus, in one aspect of this invention, a solid combination oral delivery dosage form is provided that includes an effective amount to treat a host, typically a human, of both the hemisulfate salt of Compound 1 and the di-hemisulfate salt of Compound 2.

In one embodiment, this fixed-dose combination is intended to achieve a sustained viral response in less than 12 weeks, for example less than 10 weeks, 8 weeks or 6 weeks or less. In addition to effectively treating the virus, the combination drug therapy is helpful in limiting the emergence of drug resistance.

The weight of active compound in the dosage form described herein is with respect to either the free form or the salt form of the compound unless otherwise specifically indicated. For example, 600 mg of Compound 1-A is the equivalent of 550 mg of Compound 1. The equivalent of 60 mg of Compound 2 is 67 mg of Compound 2-A and the equivalent of 100 mg of Compound 2 is 113 mg of Compound 2-A.

In a typical embodiment, Compound 1 is administered in a dosage of between about 300 and 1000 mg, more typically between 400 or 500 and 600 or 800 mg, or between 500 and 750 mg.

In one example 550 mg of Compound 1 is administered as a dosage of about 600 mg of Compound 1-A. In a typical embodiment, Compound 2 is administered in a dosage of about between 25 and 150 mg, more typically between 50 and 100 mg. In one non-limiting example, 60 mg of Compound 2 is administered in a dosage form of about 67 mg of Compound 2-A.

In various aspects, Compound 1 or its pharmaceutically acceptable salt and Compound 2 or its pharmaceutically acceptable salt, for example Compound 1-A and Compound 2-A, are formulated together in a single dosage form or provided in several dosage forms (e.g., two or more dosages, each of which has both actives or wherein one dosage has one active and the other dosage has the other active). In an alternative embodiment, Compound 1 or its pharmaceutically acceptable salt and Compound 2 or its pharmaceutically acceptable salt, are provided in separate dosage forms but in a manner that they can act in concert, for example, synergistically, in the host.

For example, the separate dosage forms can be administered such that there is an overlapping AUC, or other pharmacokinetic parameter that indicates that the actives are working together against the virus.

In one aspect of the present invention, Compound 1-A and Compound 2-A are provided in separate pills and are administered at approximately the same time over the course of a day.

The combination of Compound 1 (or a pharmaceutically acceptable salt thereof, for example Compound 1-A) and Compound 2 (or a pharmaceutically acceptable salt thereof, for example Compound 2-A) can also be used to treat related conditions such as anti-HCV antibody positive and antigen positive conditions, viral-based chronic liver inflammation, liver cancer resulting from advanced hepatitis C (hepatocellular carcinoma (HCC)), cirrhosis, chronic or acute hepatitis C, fulminant hepatitis C, chronic persistent hepatitis C and anti-HCV-based fatigue.

In certain embodiments, Compound 1 or a pharmaceutically acceptable salt thereof and Compound 2 or a pharmaceutically acceptable salt thereof, for example Compound 1-A and Compound 2-A, are administered for up to 24 weeks, up to 12 weeks, for up to 10 weeks, for up to 8 weeks, for up to 6 weeks, or for up to 4 weeks. In alternative embodiments, Compound 1 or a pharmaceutically acceptable salt thereof and Compound 2 or a pharmaceutically acceptable salt thereof, for example Compound 1-A and Compound 2-A, are administered for at least 4 weeks, for at least 6 weeks, for at least 8 weeks, for at least 10 weeks, for at least 12 weeks, or for at least 24 weeks. In certain embodiments, Compound 1 or a pharmaceutically acceptable salt thereof and Compound 2 or a pharmaceutically acceptable salt thereof are administered at least once a day or every other day.

In certain embodiments, the patient is non-cirrhotic. In certain embodiments, the patient is cirrhotic. In a further embodiment, the cirrhotic host has compensated cirrhosis. In an alternative embodiment, the cirrhotic host has decompensated cirrhosis. In one embodiment, the host has Child-Pugh A cirrhosis. In an alternative embodiment, the host has Child-Pugh B or Child-Pugh C cirrhosis.

The above combinations can also be used to treat the range of HCV genotypes. At least six distinct genotypes of HCV, each of which have multiple subtypes, have been identified globally. Genotypes 1-3 are prevalent worldwide and Genotypes 4, 5, and 6 are more limited geographically. Genotype 4 is common in the Middle East and Africa. Genotype 5 is mostly found in South Africa.

Genotype 6 predominately exists in Southeast Asia. Although the most common genotype in the United States is Genotype 1, defining the genotype and subtype can assist in treatment type and duration. For example, different genotypes respond differently to different medications. Optimal treatment times vary depending on the genotype infection. Within genotypes, subtypes, such as Genotype 1a and Genotype 1b, may respond differently to treatment as well. Infection with one type of genotype does not preclude a later infection with a different genotype.

In one embodiment, a combination of Compound 1 or a pharmaceutically acceptable salt thereof and Compound 2 or a pharmaceutically acceptable salt thereof, for example Compound 1-A and Compound 2-A, are used to treat HCV Genotype 1, HCV Genotype 2, HCV Genotype 3, HCV Genotype 4, HCV Genotype 5, or HCV Genotype 6. In one embodiment, Compound 1 or a pharmaceutically acceptable salt thereof and Compound 2 or a pharmaceutically acceptable salt thereof are used to treat HCV Genotype 1a. In one embodiment, Compound 1 or a pharmaceutically acceptable salt thereof and Compound 2 or a pharmaceutically acceptable salt thereof are used to treat HCV Genotype 1b. In one embodiment, Compound 1 or a pharmaceutically acceptable salt thereof and Compound 2 or a pharmaceutically acceptable salt thereof are used to treat HCV Genotype 2a. In one embodiment, Compound 1 or a pharmaceutically acceptable salt thereof and Compound 2 or a pharmaceutically acceptable salt thereof are used to treat HCV Genotype 2b. In one embodiment, Compound 1 or a pharmaceutically acceptable salt thereof and Compound 2 or a pharmaceutically acceptable salt thereof are used to treat HCV Genotype 3a. In one embodiment, Compound 1 or a pharmaceutically acceptable salt thereof and Compound 2 or a pharmaceutically acceptable salt thereof are used to treat HCV Genotype 3b. In one embodiment, Compound 1 or a pharmaceutically acceptable salt thereof and Compound 2 or a pharmaceutically acceptable salt thereof are used to treat HCV Genotype 4a. In one embodiment, Compound 1 or a pharmaceutically acceptable salt thereof and Compound 2 or a pharmaceutically acceptable salt thereof are used to treat HCV Genotype 4d. In one embodiment, Compound 1 or a pharmaceutically acceptable salt thereof and Compound 2 or a pharmaceutically acceptable salt thereof are used to treat HCV Genotype 5a. In one embodiment, Compound 1 or a pharmaceutically acceptable salt thereof and Compound 2 or a pharmaceutically acceptable salt thereof are used to treat HCV Genotype 6a. In one embodiment, Compound 1 or a pharmaceutically acceptable salt thereof and Compound 2 or a pharmaceutically acceptable salt thereof are used to treat HCV Genotype 6b, 6c, 6d, 6e, 6f, 6g, 6h, 6i, 6j, 6k, 6l, 6m, 6n, 6o, 6p, 6q, 6r, 6s, 6t, or 6u.

In one embodiment, a combination of Compound 1-A and Compound 2-A are used to treat HCV Genotype 1, HCV Genotype 2, HCV Genotype 3, HCV Genotype 4, HCV Genotype 5, or HCV Genotype 6. In one embodiment, Compound 1-A and Compound 2-A are used to treat HCV Genotype 1a. In one embodiment, Compound 1-A and Compound 2-A are used to treat HCV Genotype 1b. In one embodiment, Compound 1-A and Compound 2-A are used to treat HCV Genotype 2a. In one embodiment, Compound 1-A and Compound 2-A are used to treat HCV Genotype 2b. In one embodiment, Compound 1-A and Compound 2-A are used to treat HCV Genotype 3a. In one embodiment, Compound 1-A and Compound 2-A are used to treat HCV Genotype 4a. In one embodiment, Compound 1-A and Compound 2-A are used to treat HCV Genotype 4d.

In one embodiment, Compound 1-A and Compound 2-A are used to treat HCV Genotype 5a. In one embodiment, Compound 1-A and Compound 2-A are used to treat HCV Genotype 6a. In one embodiment, Compound 1-A and Compound 2-A are used to treat HCV Genotype 6b, 6c, 6d, 6e, 6f, 6g, 6h, 6i, 6j, 6k, 6l, 6m, 6n, 6o, 6p, 6q, 6r, 6s, 6t, or 6u.

The invention also includes the specific combination and dosage forms wherein Compound 1-A may be in the form of an amorphous or crystalline salt and, independently, Compound 2-A may be crystalline or amorphous.

The present invention thus includes at least the following embodiments:
(a) An effective combination of Compound 1 or a pharmaceutically acceptable salt thereof and Compound 2 or a pharmaceutically acceptable salt thereof for the treatment of an HCV-infected patient, typically a human.
(b) An effective solid dosage form of a combination of Compound 1 or a pharmaceutically acceptable salt thereof and Compound 2 or a pharmaceutically acceptable salt thereof for treatment of an HCV-infected patient, typically a human (c) An effective combination of Compound 1-A and Compound 2-A for treatment of an HCV-infected patient, typically a human.
(d) A solid dosage form of a combination of Compound 1-A and Compound 2-A for treatment of an HCV-infected patient, typically a human.
(e) Embodiment (a) or (b) wherein Compound 2 or a pharmaceutically acceptable salt thereof is Compound 2-B.
(f) Embodiment (a) or (b) wherein Compound 2 or a pharmaceutically acceptable salt thereof is Compound 2-C.
(g) Any of embodiments (a)-(f) wherein the combination is in the form of a combined pharmaceutical composition form.
(h) Any of embodiments (a)-(f) wherein the combination is in the form of separate pharmaceutical dosage forms for each anti-HCV active agent, for use in a concerted fashion.
(i) The pharmaceutical dosage forms of (g) or (h) which are suitable for oral delivery.
(j) The dosage form of (g) that is in the form of a pill, tablet, or gel.
(k) The pharmaceutical dosage forms of (g) or (h) which are suitable for parental delivery.
(l) The pharmaceutical dosage forms of (g) or (h) which are suitable for intravenous delivery.
(m) Any one of embodiments (a)-(k), wherein Compound 1, Compound 1-A, Compound 2, or Compound 2-A is in a morphic form.
(n) Compound 2-A of the formula:

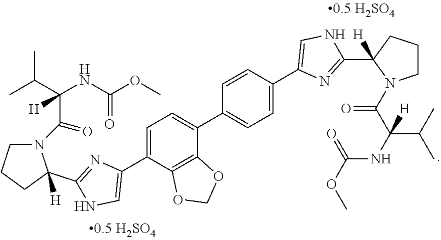 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

(o) An isolated morphic form of Compound 2-A described herein characterized by an XRPD pattern that is substantially similar to the XRPD pattern of FIG. 1A.
(p) An isolated morphic form of Compound 2-A described herein characterized by an XRPD pattern comprising 20 values including at least or selected from 7.3+/−0.2°2θ, 7.9+/−0.2°2θ, 12.0+/−0.2°2θ, 12.2+/−0.2°2θ, 14.7+/−0.2°2θ, 15.8+/−0.2°2θ, 16.1+/−0.2°2θ, 16.5+/−0.2°2θ, 18.2+/−0.2°2θ, and 22.7+/−0.2°2θ.
(q) Embodiment (p) wherein the standard deviation is +/−0.3°2θ.
(r) Embodiment (p) wherein the standard deviation is +/−0.4°2θ.
(s) Embodiment (m), wherein Compound 2-A is in a morphic form.
(t) Embodiment (m), wherein Compound 2-A is in a morphic form characterized by an XRPD pattern substantially similar to FIG. 1A.
(u) Embodiment (m), wherein Compound 2-A is in a morphic form characterized by an XRPD pattern comprising 20 values including at least or selected from 7.3+/−0.2°2θ, 7.9+/−0.2°2θ, 12.0+/−0.2°2θ, 12.2+/−0.2°2θ, 14.7+/−0.2°2θ, 15.8+/−0.2°2θ, 16.1+/−0.2°2θ, 16.5+/−0.2°2θ, 18.2+/−0.2°2θ, and 22.7+/−0.2°2θ.
(v) Embodiment (u) wherein the standard deviation is +/−0.3°2θ.
(w) Embodiment (u) wherein the standard deviation is +/−0.4°2θ.
(x) Any one of the above embodiments, wherein an additional anti-HCV effective compound is used in the combination.
(y) A pharmaceutical composition comprising any one of embodiments (a)-(x) and a pharmaceutically acceptable excipient.
(z) A pharmaceutical composition comprising any one of embodiments (a)-(x) and a pharmaceutically acceptable excipient and a third anti-HCV effective agent wherein the third anti-HCV an effective agent acts through a different mechanism than Compound 1, Compound 1-A, Compound 2, or Compound 2-A.
(aa) Use of an effective combination of any one of embodiments (a)-(z) in the manufacture of a medicament for treatment of a hepatitis C virus infection in a patient in need thereof.
(bb) A method for manufacturing a medicament intended for the therapeutic use for treating a hepatitis C virus infection in a patient in need thereof, characterized in that an effective combination of any one of embodiments (a)-(z) is used in the manufacture.
(cc) A method for treating a hepatitis C virus infection comprising administering to a patient in need thereof an effective combination of any one of embodiments (a)-(z).
(dd) A method for curing a hepatitis C virus infection comprising administering to a patient in need thereof an effective combination of any one of embodiments (a)-(z).
(ee) A method for prophylactically treating a patient at risk of a hepatitis C virus infection comprising administering to the patient in need thereof an effective combination of any one of embodiments (a)-(z).
(ff) A method for treating conditions related to a hepatitis C viral infection selected from viral-based chronic liver inflammation, liver cancer resulting from advanced hepatitis C (hepatocellular carcinoma (HCC)), cirrhosis, chronic or acute hepatitis C, fulminant hepatitis C, chronic persistent hepatitis C and anti-HCV-based fatigue comprising administering to the patient in a patient in need thereof an effective combination of any one of embodiments (a)-(z).
(gg) Any of embodiments (aa)-(ff) wherein the patient is cirrhotic.
(hh) Any of embodiments (aa)-(ff) wherein the patient is non-cirrhotic.
(ii) Any of embodiments (aa)-(hh) wherein the HCV infection is genotype 1.
(jj) Any of embodiments (aa)-(hh) wherein the HCV infection is genotype 2.
(kk) Any of embodiments (aa)-(hh) wherein the HCV infection is genotype 3.
(ll) Any of embodiments (aa)-(hh) wherein the HCV infection is genotype 4.
(mm) Any of embodiments (aa)-(hh) wherein the HCV infection is genotype 5.
(nn) Any of embodiments (aa)-(hh) wherein the HCV infection is genotype 6.

### BRIEF DESCRIPTION OF FIGURES

FIG. 1A is an XRPD pattern for Compound 2-A as described in Example 3. The x-axis is two-theta measured in degrees and the y-axis is intensity measured in counts.

FIG. 1B is a DSC graph for Compound 2-A as described in Example 3. The upper x-axis is temperature measured in Celsius and the lower x-axis is time measured in minutes. The y-axis is weight measured in mg.

FIG. 2 is an XRPD pattern for Compound 2-B as described in Example 3. The x-axis is two-theta measured in degrees and the y-axis is intensity measured in counts.

FIG. 3 is an XRPD pattern for Compound 2-C as described in Example 3. The x-axis is two-theta measured in degrees and the y-axis is intensity measured in counts.

FIG. 4A is an isobologram for 90% inhibition of HCV genotype 1a (GT1a) using the combination of Compound 1-A and Compound 2 as described in Example 6. The x-axis represents the concentration in nM of Compound 2 required to achieve 90% inhibition of HCV GT1a and the y-axis represents the concentration in nM of Compound 1-A required to achieve 90% inhibition of HCV GT1a. A line of additivity is formed by connecting the dose at which Compound 1-A alone achieves 90% inhibition with the dose at which point Compound 2 achieves 90% inhibition. The star (*) on the graph represents the concentrations of the two drugs together that achieve 90% inhibition. The star is below the line of additivity, indicating that a synergistic effect is observed for the combination of Compound 1-A and Compound 2 against HCV GT1a.

FIG. 4B is an isobologram for 90% inhibition of HCV genotype 1b (GT1b) using the combination of Compound 1-A and Compound 2 as described in Example 6. The x-axis represents the concentration in nM of Compound 2 required to achieve 90% inhibition of HCV GT1b and the y-axis represents the concentration in nM of Compound 1-A required to achieve 90% inhibition of HCV GT1b. A line of additivity is formed by connecting the dose at which Compound 1-A alone achieves 90% inhibition with the dose at which point Compound 2 achieves 90% inhibition. The star (*) on the graph represents the concentrations of the two drugs together that achieve 90% inhibition. The star is below the line of additivity, indicating that a synergistic effect is observed for the combination of Compound 1-A and Compound 2 against HCV GT1b.

FIG. 4C is an isobologram for 90% inhibition of chimeric HCV replicons containing the GT3a-NS5B genotype (GT1b_3a-NS5a) using the combination of Compound 1-A and Compound 2 as described in Example 6. The x-axis represents the concentration in nM of Compound 2 required to achieve 90% inhibition of the chimeric HCV replicons and the y-axis represents the concentration in nM of Compound 1-A required to achieve 90% inhibition of chimeric HCV replicons. A line of additivity is formed by connecting the dose at which Compound 1-A alone achieves 90% inhibition with the dose at which point Compound 2 achieves 90% inhibition. The star (*) on the graph represents the concentrations of the two drugs together that achieve 90% inhibition. The star is below the line of additivity, indicating that a synergistic effect is observed for the combination of Compound 1-A and Compound 2 against the chimeric HCV replicons containing GT1b_3a-NS5B.

FIG. 5 is the NS5B polymerase inhibitor Compound 1-A and the NS5A inhibitor Compound 2-A.

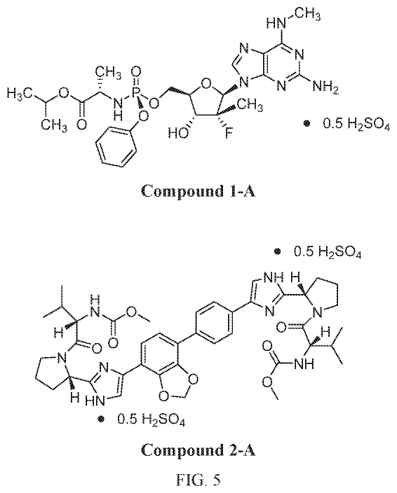

### DETAILED DESCRIPTION OF THE INVENTION

The present invention provides a highly active combination of a specific NS5B polymerase inhibitor and a specific NS5A inhibitor for the advantageous treatment of a hepatitis C infection in a host, typically a human.

The anti-HCV compounds used in this combination therapy are: 1) the NS5B inhibitor isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate (Compound 1), or a pharmaceutically acceptable salt thereof and 2) the NS5A inhibitor methyl N-[(2S)-1-[(2S)-2-[5-[4-[7-[2-[(2S)-1-[(2S)-2-(methoxycarbonylamino)-3-methylbutanoyl]pyrrolidin-2-yl]-1H-imidazol-5-yl]-1,3-benzodioxol-4-yl]phenyl]-1H-imidazol-2-yl]pyrrolidin-1-yl]-3-methyl-1-oxobutan-2-yl]carbamate) (Compound 2), or a pharmaceutically acceptable salt thereof. In a typical embodiment, Compound 1 is administered as the hemi-sulfate salt derivative (Compound 1-A). In a typical embodiment, Compound 2 is administered as the di-hemisulfate salt derivative (Compound 2-A).

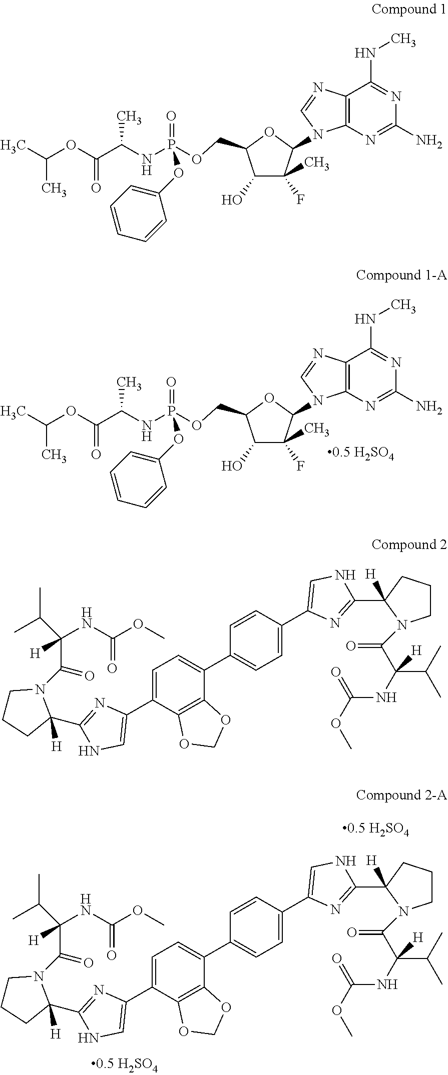 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F ; CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F ; COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C ; COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

In one embodiment, the combination of drugs are administered in a fixed-dose dosage form, such as a pill or tablet. In an alternative embodiment, the two compounds are administered in a manner that the host in need thereof receives the benefit of the both compounds in a concerted fashion, as measured by standard pharmacokinetics.

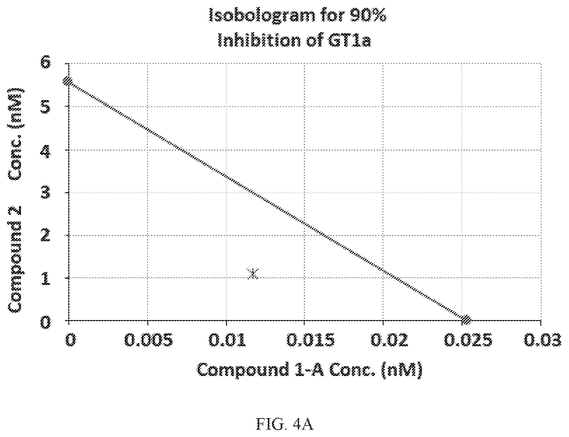

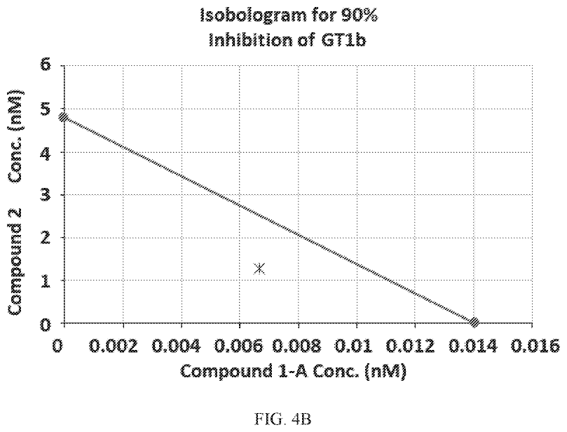

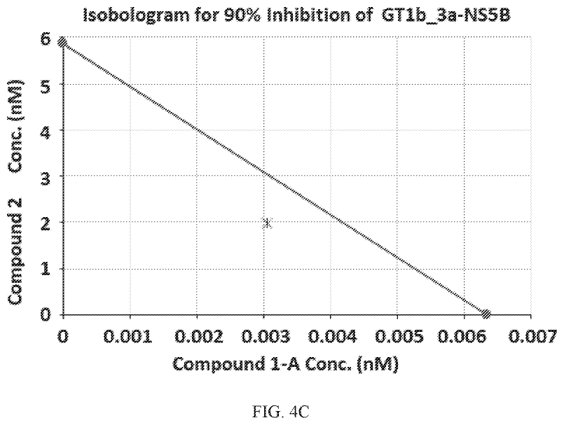

It has been unexpectedly discovered that the combination of Compound 1 and Compound 2 acts synergistically to provide an optimal anti-HCV therapeutic effect (Example 6, FIG. 4A-FIG. 4C). Two drugs in a combination regimen can be antagonistic, additive, or synergistic, and it cannot be predicted in advance how two active drugs will interact when administered to a human.

Thus, it has been surprisingly discovered that Compound 1 and Compound 2 show synergistic activity against the hepatitis C virus Co-formulated drugs for HCV tend to comprise no salts or only one salt. It is unusual for a stable, solid combination dosage form to comprise two salts because this might risk increasing the hygroscopicity or stability of the dosage form. However, in the present invention, Compound 1-A and Compound 2-A are be formulated together, perhaps due to the advantageous properties of crystalline Compound 2-A.

Compound 1 (isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate) was previously described in U.S. Pat. Nos. 9,828,410; 10,000,523; 10,005,811; and, 10,239,911 and PCT Applications WO 2016/21276 and WO 2019/200005 assigned to Atea Pharmaceuticals. The synthesis of Compound 1 is described in Example 1 below.

Compound 1-A was previously disclosed in US 2018-0215776 and PCT Applications WO 2018/144640 and WO 2019/200005 assigned to Atea Pharmaceuticals. The synthesis of Compound 1-A (the hemi-sulfate salt of isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate) is described in Example 2 below. In one embodiment Compound 1-A is provided in a pharmaceutically acceptable composition or solid dosage form thereof. In another embodiment, Compound 1-A is an amorphous solid. In one embodiment, Compound 1-A is a crystalline solid.

A non-limiting illustrative process for the preparation of Compound 1-A includes
(i) a first step of dissolving Compound 1 in an organic solvent, for example, acetone, ethyl acetate, methanol, acetonitrile, or ether, or the like, in a flask or container;
(ii) charging a second flask or container with a second organic solvent, which may be the same as or different from the organic solvent in step (i), optionally cooling the second solvent to 0-10 degrees C., and adding dropwise H₂SO₄ to the second organic solvent to create a H₂SO₄/organic solvent mixture; and wherein the solvent for example may be methanol;
(iii) adding dropwise the H₂SO₄/solvent mixture at a molar ratio of 0.5/1.0 from step (ii) to the solution of Compound 1 of step (i) at ambient or slightly increased or decreased temperature (for example 23-35 degrees C.);
(iv) stirring the reaction of step (iii) until precipitate of Compound 1-A is formed, for example at ambient or slightly increased or decreased temperature;
(v) optionally filtering the resulting precipitate from step (iv) and washing with an organic solvent; and
(vi) optionally drying the resulting Compound 1-A in a vacuum, optionally at elevated a temperature, for example, 55, 56, 57, 58, 59, or 60° C.

In certain embodiments, step (i) above is carried out in acetone. Further, the second organic solvent in step (ii) may be for example methanol and the mixture of organic solvents in step (v) is methanol/acetone.

In one embodiment, Compound 1 is dissolved in ethyl acetate in step (i). In one embodiment, Compound 1 is dissolved in tetrahydrofuran in step (i). In one embodiment, Compound 1 is dissolved in acetonitrile in step (i). In an additional embodiment, Compound 1 is dissolved in dimethylformamide in step (i).

In one embodiment, the second organic solvent in step (ii) is ethanol. In one embodiment, the second organic solvent in step (ii) is isopropanol. In one embodiment, the second organic solvent in step (ii) is n-butanol.

In one embodiment, a mixture of solvents are used for washing in step (v), for example, ethanol/acetone. In one embodiment, the mixture of solvent for washing in step (v) is isopropanol/acetone. In one embodiment, the mixture of solvent for washing in step (v) is n-butanol/acetone. In one embodiment, the mixture of solvent for washing in step (v) is ethanol/ethyl acetate. In one embodiment, the mixture of solvent for washing in step (v) is isopropanol/ethyl acetate. In one embodiment, the mixture of solvent for washing in step (v) is n-butanol/ethyl acetate. In one embodiment, the mixture of solvent for washing in step (v) is ethanol/tetrahydrofuran. In one embodiment, the mixture of solvent for washing in step (v) is isopropanol/tetrahydrofuran. In one embodiment, the mixture of solvent for washing in step (v) is n-butanol/tetrahydrofuran. In one embodiment, the mixture of solvent for washing in step (v) is ethanol/acetonitrile. In one embodiment, the mixture of solvent for washing in step (v) is isopropanol/acetonitrile. In one embodiment, the mixture of solvent for washing in step (v) is n-butanol/acetonitrile. In one embodiment, the mixture of solvent for washing in step (v) is ethanol/dimethylformamide. In one embodiment, the mixture of solvent for washing in step (v) is isopropanol/dimethylformamide. In one embodiment, the mixture of solvent for washing in step (v) is n-butanol/dimethylformamide.

Compound 1-A has completed a Phase 1b/2a clinical trial for patients infected with HCV. The multiple part study evaluated the effect of single and multiple doses of Compound 1-A in healthy subjects, non-cirrhotic HCV-infected patients, and cirrhotic HCV-infected patients. Compound 1-A induced significant antiviral reduction when administered to all HCV-infected cohorts tested. Compound 1-A was administered once daily (QD) over the course of seven days, and potent antiviral activity was observed. In non-cirrhotic HCV-infected patients who were given 600 mg QD of Compound 1-A (equivalent to 550 mg of Compound 1), the mean maximum HCV RNA reduction was 4.4 log₁₀ IU/mL in HCV GT1-infected patients and 4.6 log₁₀ IU/mL in HCV GT3-infected patients. The effect of Compound 1-A on antiviral reduction also extended to the difficult-to-treat cirrhotic patients. In a cohort of HCV GT1 or HCV GT3-infected patients with CPA cirrhosis, the mean maximum HCV RNA reduction was 4.4 log₁₀ IU/mL when administered QD for seven days (Zhou, X. et al. “AT-527, a pan-genotypic purine nucleotide prodrug, exhibits potent antiviral activity in subjects with chronic hepatitis C” presented at The International Liver Congress 2018; Apr. 13, 2018; Paris, France).

Unless otherwise specified, Compound 1 or a pharmaceutically acceptable salt thereof, for example, Compound 1-A, is provided in the β-D-configuration. In an alternative embodiment, Compound 1 or a pharmaceutically acceptable salt thereof, for example Compound 1-A, can be provided in a 3-L-configuration. The phosphoramidate of Compound 1 or a pharmaceutically acceptable salt thereof, for example Compound 1-A, can be provided as an R or S chiral phosphorus derivative or a mixture thereof, including a racemic or a diastereomeric mixture. All of the combinations of these stereoconfigurations are alternative embodiments in the invention described herein.

These alternative configurations include, but are not limited to:

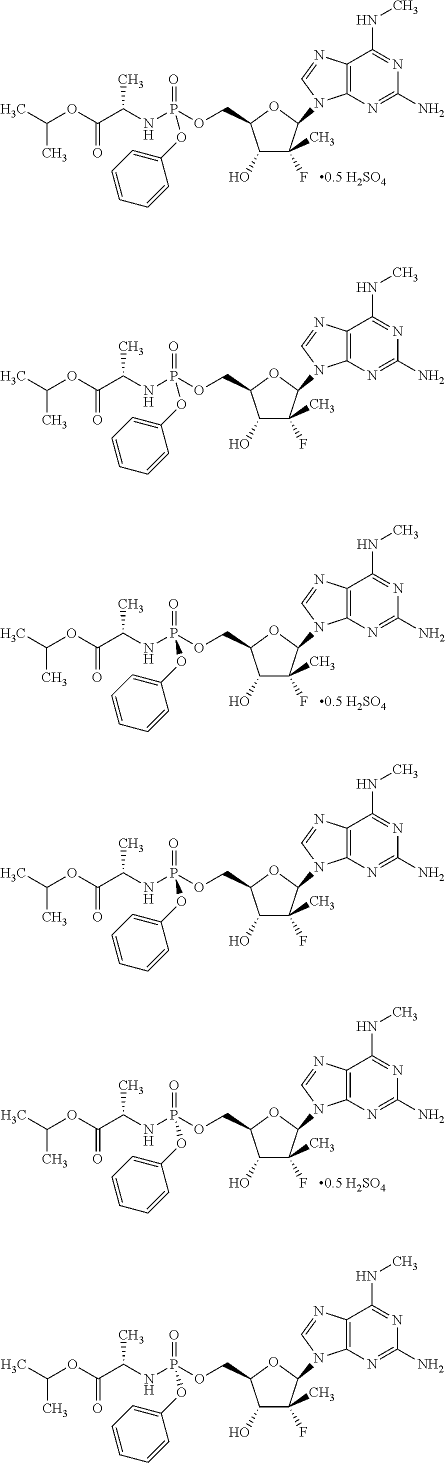

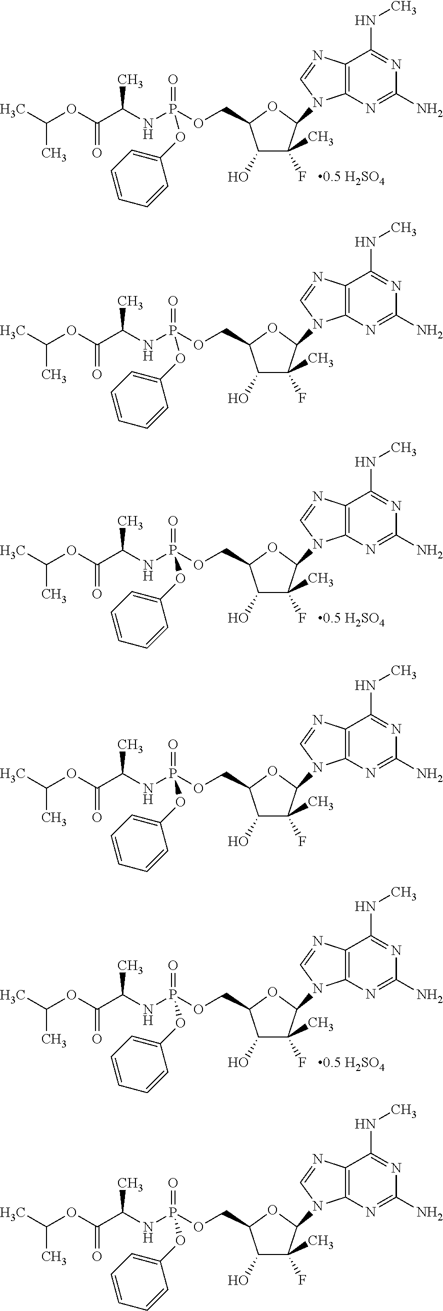

An additional alternative configuration includes

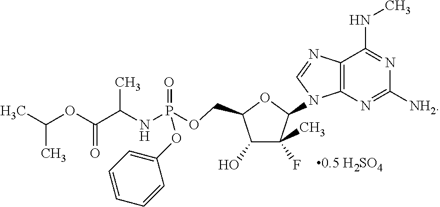 [SMILES: CNc1nc(N)nc2c1ncn2[C@@H]1O[C@H](COP(=O)(NC(C)C(=O)OC(C)C)Oc2ccccc2)[C@@H](O)[C@@]1(C)F]

In one embodiment, any of the above stereoisomers or a pharmaceutically acceptable salt thereof is used as Compound 1 in any aspect of the present invention herein. In another embodiment any one of the above stereoisomers or a pharmaceutically acceptable salt thereof is used as Compound 1-A in any aspect of the present invention herein.

In an alternative embodiment, Compound 1-A is provided as the hemisulfate salt of a phosphoramidate other than the specific phosphoramidate described in the compound illustration. In another alternative embodiment, Compound 1 or a pharmaceutically acceptable salt thereof is provided as a phosphoramidate other than the specific phosphoramidate described in the compound illustration. A wide range of phosphoramidates are known to those skilled in the art which can be selected as desired to provide an active compound as described herein. For example, the phosphoramidate of Compound 1 or a pharmaceutically acceptable salt thereof includes a compound or pharmaceutically acceptable salt thereof of Formula A:

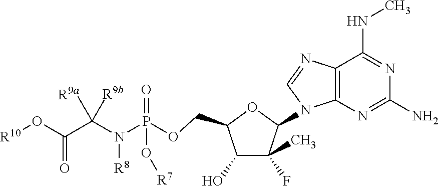

wherein:

R⁷ is hydrogen, C₁₋₆alkyl (including methyl, ethyl, propyl, and isopropyl), C₃₋₇cycloalkyl, or aryl (including phenyl and napthyl);

R⁸ is hydrogen or C₁₋₆alkyl (including methyl, ethyl, propyl, and isopropyl);

R^{9a} and R^{9b} are independently selected from hydrogen, C₁₋₆alkyl (including methyl, ethyl, propyl, and isopropyl), or C₃₋₇cycloalkyl; and

R¹⁰ is hydrogen, C₁₋₆alkyl (including methyl, ethyl, propyl, and isopropyl), C₁₋₆haloalkyl, or C₃₋₇cycloalkyl.

In alternative non-limiting embodiments, the present invention includes Compound 1 as an oxalate salt (Compound 1-B), an HCl salt (Compound 1-C), or a sulfate salt (Compound 1-D).

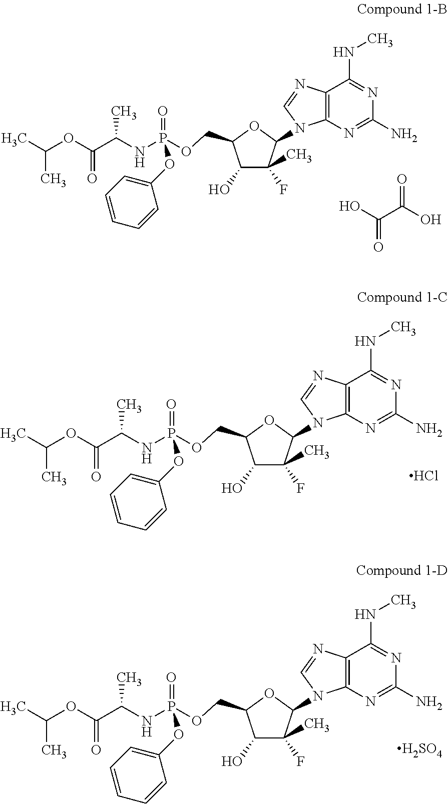

The metabolism of Compound 1 and Compound 1-A involves the production of a 5′-monophosphate and the subsequent anabolism of the N⁶-methyl-2,6-diaminopurine base (1-3) to generate ((2R,3R,4R,5R)-5-(2-amino-6-oxo-1,6-dihydro-9H-purin-9-yl)-4-fluoro-3-hydroxy-4-methyltetrahydrofuran-2-yl)methyl dihydrogen phosphate (1-4) as the 5′-monophosphate. The monophosphate is then further anabolized to the active triphosphate species: the 5′-triphosphate (1-6). The 5′-triphosphate can be further metabolized to generate 2-amino-9-((2R,3R,4R,5R)-3-fluoro-4-hydroxy-5-(hydroxymethyl)-3-methyltetrahydrofuran-2-yl)-1,9-dihydro-6H-purin-6-one (1-7). Alternatively, 5′-monophophate 1-2 can be metabolized to generate the purine base 1-8. The metabolic pathway for isopropyl((S)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate is illustrated in Scheme 1:

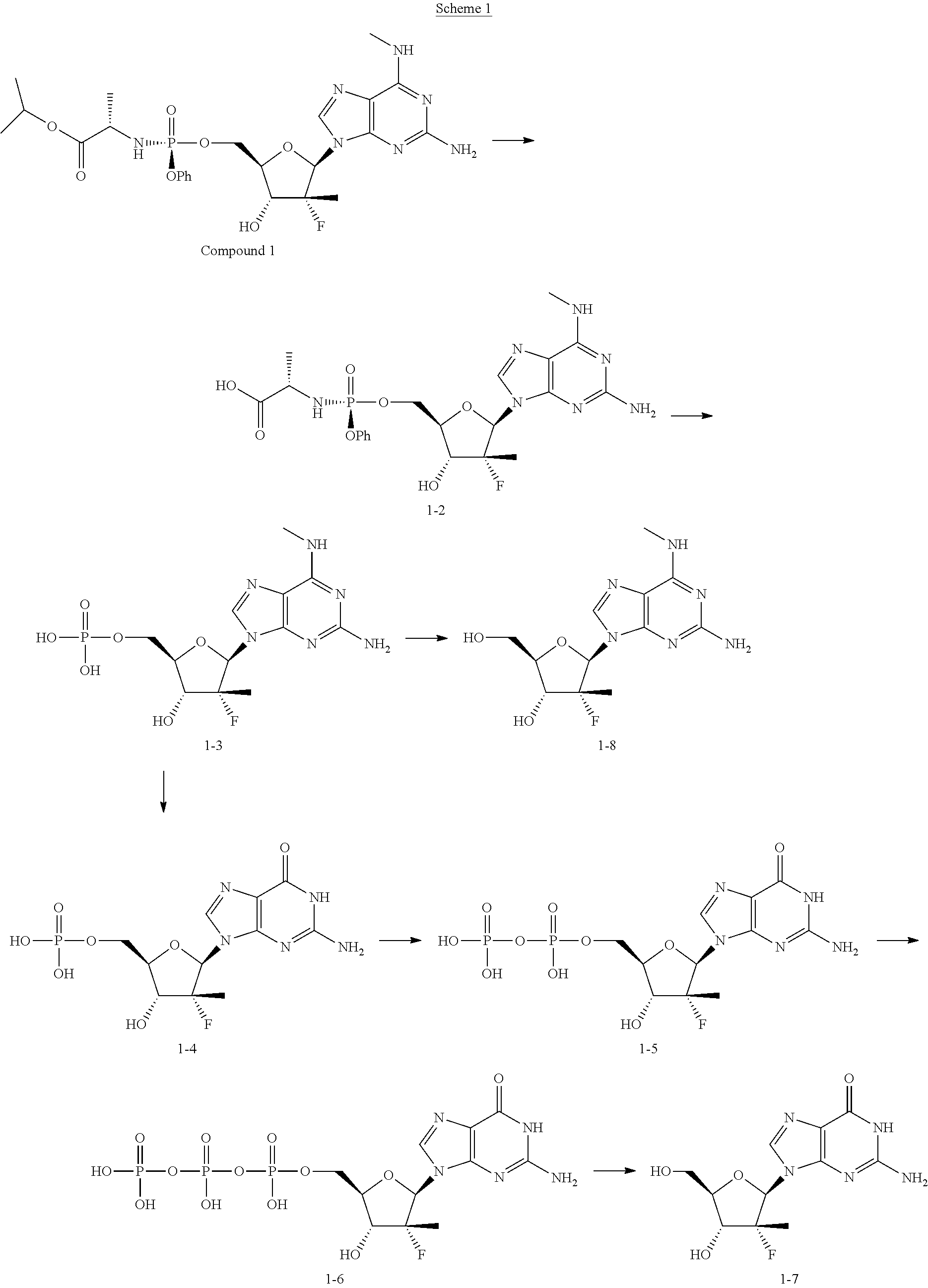

Atea Pharmaceuticals, Inc. has disclosed 3-D-2′-deoxy-2′-α-fluoro-2′-β-C-substituted-2-modified-N⁶-(mono- and di-methyl) purine nucleotides for the treatment of HCV in U.S. Pat. Nos. 9,828,410; 10,000,523; 10,005,811; and, 10,239,911 and US2018-0215776; and, PCT Application Nos. WO 2016/144918; WO 2018/048937; WO 2018/013937; and, WO 2018/144640. Atea has also disclosed β-D-2′-deoxy-2′-substituted-4′-substituted-2-N⁶-substituted-6-aminopurine nucleotides for the treatment of paramyxovirus and orthomyxovirus infections in U.S. Pat. No. 10,202,412 and PCT Application No. WO 2018/009623.

### Compound 2 and Compound 2-A

Compound 2 is disclosed in WO 2011/075607 and U.S. Patent Application US 2011/0152246 (page 104) assigned to InterMune, Inc.

In one embodiment, Compound 2 is administered as the pharmaceutically acceptable salt thereof, for example Compound 2-A. In one embodiment a solid form of Compound 2 or 2-A is used. In one embodiment the solid form of Compound 2 or 2-A is a crystalline solid.

The synthesis of Compound 2 (Coblopasvir or KW-136; methyl N-[(2S)-1-[(2S)-2-[5-[4-[7-[2-[(2 S)-1-[(2 S)-2-(methoxycarbonylamino)-3-methylbutanoyl]pyrrolidin-2-yl]-1H-imidazol-5-yl]-1,3-benzodioxol-4-yl]phenyl]-1H-imidazol-2-yl]pyrrolidin-1-yl]-3-methyl-1-oxobutan-2-yl]carbamate) is known in the art. Non-limiting examples of synthetic methods that can be used to prepare Compound 2 include those reported in WO 2011/075607 assigned to InterMune, Inc.

A crystalline form and formulation of Compound 2 are described in Chinese Patent Applications Nos. CN 108904496 and CN 108675998 assigned to Beijing Kawin Technology Share-Holding Co. To date, the only crystalline form of Compound 2 that has been previously disclosed is the di-HCl salt as described in Chinese Patent Applications '496 and '998.

The present invention provides a new salt form of Compound 2, the di-hemisulfate Compound 2-A, and an advantageous isolated morphic form of Compound 2-A.

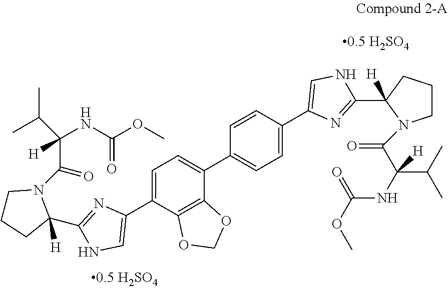 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

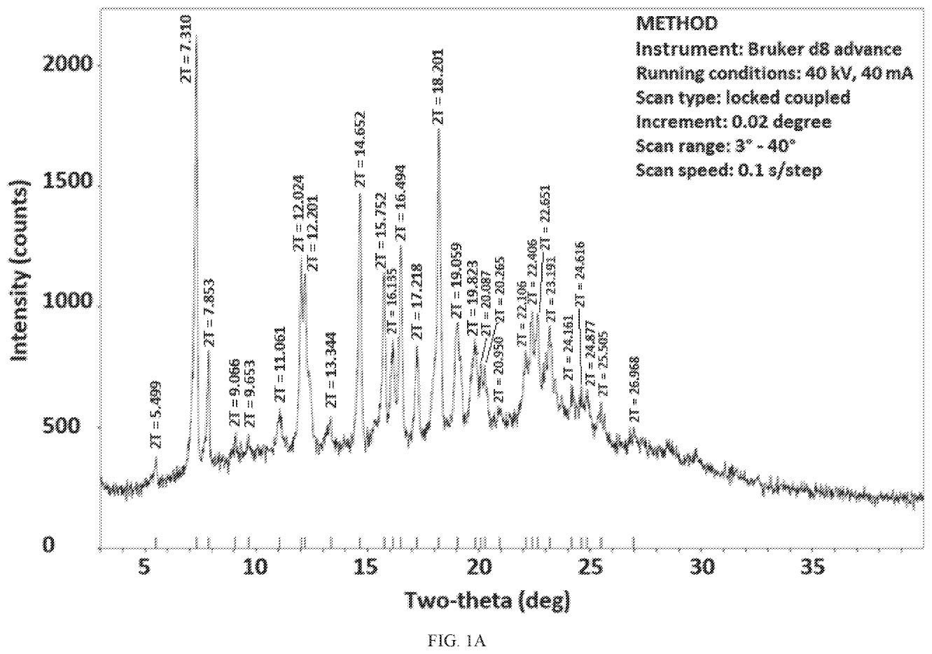

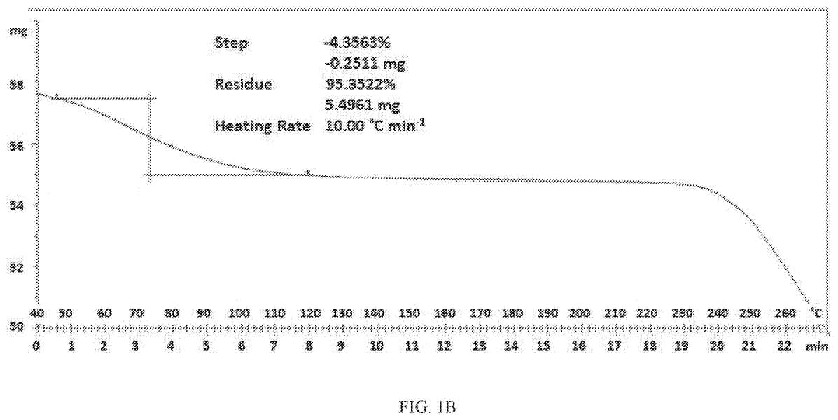

In one embodiment, the morphic form of Compound 2-A is characterized by an XRPD pattern that is substantially similar to that set forth in FIG. 1A. In one embodiment, the morphic form of Compound 2-A is characterized by an XRPD pattern comprising at least five, at least six, at least seven, at least eight, at least nine, or at least ten 20 values from Table 2. In one embodiment, the morphic form of Compound 2-A is characterized by an XRPD pattern comprising:
a) 20 values including at least or selected from 7.3, 7.9, 12.0, 12.2, 14.7, 15.8, 16.1, 16.5, 18.2, and 22.7+/−0.2°2θ;
b) at least two, three, or four 20 values selected from 7.3, 7.9, 12.0, 12.2, 14.7, 15.8, 16.1, 16.5, 18.2, and 22.7+/−0.2°2θ;
c) at least five, six, or seven 20 values selected from 7.3, 7.9, 12.0, 12.2, 14.7, 15.8, 16.1, 16.5, 18.2, and 22.7+/−0.2°2θ;
d) at least eight or nine 20 values selected from 7.3, 7.9, 12.0, 12.2, 14.7, 15.8, 16.1, 16.5, 18.2, and 22.7+/−0.2°2θ;
e) 20 values including at least or selected from 7.3, 12.0, 14.7, 16.5, and 18.2+/−0.2°2θ; or
f) at least one 20 value selected from 7.3, 12.0, 14.7, 16.5, and 18.2+/−0.2°2θ.

The plus-minus notation “+/−0.2°2θ” as used to describe morphic forms refers to 20 values in the list characterized by +/−0.2°2θ. For example, in (a) above, 20 values including at least or selected from 7.3, 7.9, 12.0, 12.2, 14.7, 15.8, 16.1, 16.5, 18.2, and 22.7+/−0.2°2θ includes independently the following 20 values 7.3+/−0.2°2θ, 7.9+/−0.2°2θ, 12.0+/−0.2°2θ, 12.2+/−0.2°2θ, 14.7+/−0.2°2θ, 15.8+/−0.2°2θ, 16.1+/−0.2°2θ, 16.5+/−0.2°2θ, 18.2+/−0.2°2θ, and 22.7+/−0.2°2θ. In an alternative embodiment, the standard deviation is +/−0.3°2θ. In an alternative embodiment, the standard deviation is +/−0.4°2θ. The standard deviation of +/−0.2°2θ as used to describe morphic forms also includes the standard deviation of +/−0.3°2θ and +/−0.4°2θ.

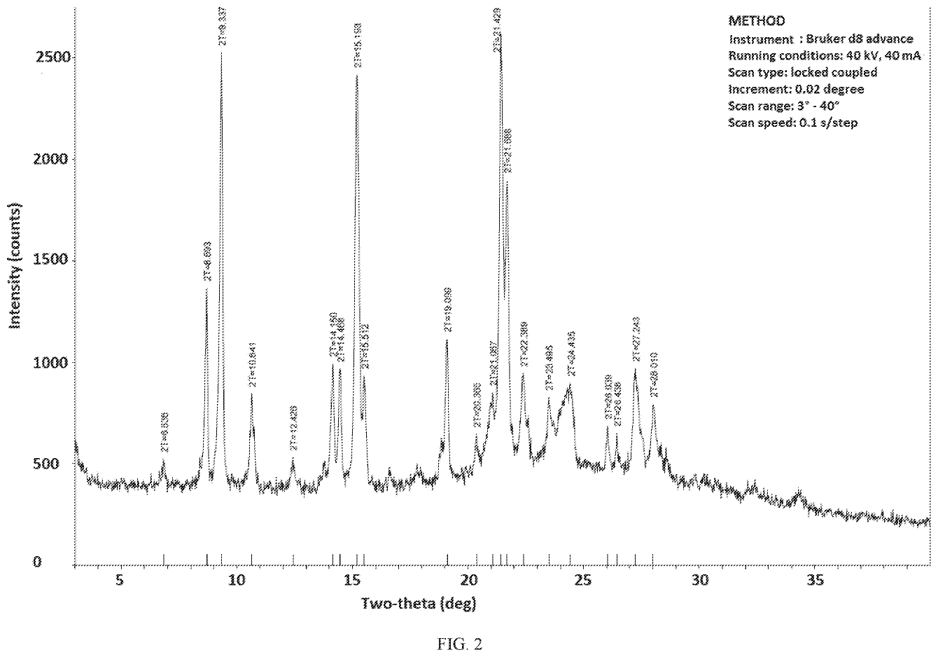

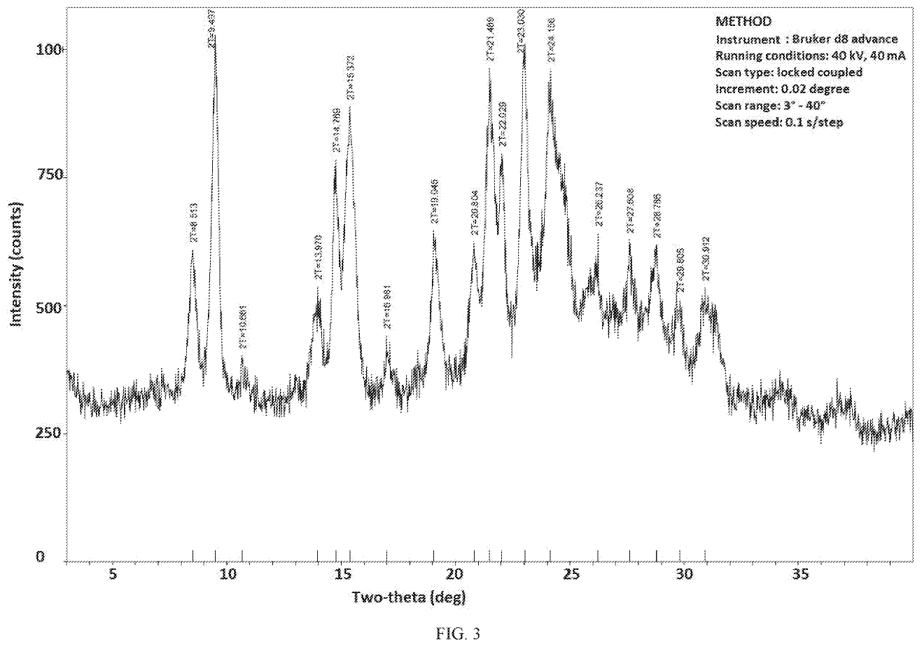

The crystallization investigation of Example 3 also resulted in two additional solid, crystalline salt forms of Compound 2, the di-nitrate salt (Compound 2-B) and the di-hydrobromate salt (Compound 2-C). These crystallization forms were very solvent-specific. Compound 2-B was only a crystalline solid when tested in CH₃CN despite studies in four other solvents and CH₃CN is not preferred for pharmaceutical uses. Compound 2-C was only a crystalline solid in i-PrOH, but not H₂O. The XRPD pattern of the isolated morphic form of the di-nitrate salt of Compound 2 is provided in FIG. 2 and the XRPD pattern of the isolated morphic form of the di-hydrobromate of Compound 2 is provided in FIG. 3.

In an alternative embodiment, Compound 2 is administered as the pharmaceutically acceptable di-nitrate salt, Compound 2-B.

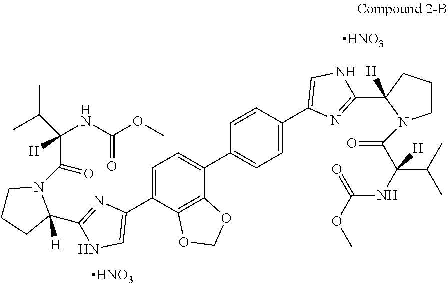

The present invention also describes the morphic form of the di-nitrate salt of Compound 2, Compound 2-B. In one embodiment, the morphic form of Compound 2-B is characterized by an XRPD pattern that is substantially similar to that set forth in FIG. 2. In one embodiment, the morphic form of Compound 2-B is characterized by an XRPD pattern comprising at least five, at least six, at least seven, at least eight, at least nine, or at least ten 20 values from Table 3. In one embodiment, the morphic form of Compound 2-B is characterized by an XRPD pattern comprising:
a) 20 values including at least or selected from 8.7, 9.3, 14.2, 14.7, 15.2, 15.5, 19.1, 21.4, 21.7, and 27.2+/−0.2°2θ;
b) at least two, three, or four 20 values selected from 8.7, 9.3, 14.2, 14.7, 15.2, 15.5, 19.1, 21.4, 21.7, and 27.2+/−0.2°2θ;
c) at least five, six, or seven 20 values selected from 8.7, 9.3, 14.2, 14.7, 15.2, 15.5, 19.1, 21.4, 21.7, and 27.2+/−0.2°2θ;
d) at least eight or nine 20 values selected from 8.7, 9.3, 14.2, 14.7, 15.2, 15.5, 19.1, 21.4, 21.7, and 27.2+/−0.2°2θ;
e) 20 values including at least or selected from 8.7, 9.3, 15.2, 21.4, and 21.7+/−0.2°2θ; or
f) at least one 20 value selected from 8.7, 9.3, 15.2, 21.4, and 21.7+/−0.2°2θ.

In an alternative embodiment, the standard deviation is +/−0.3°2θ. In an alternative embodiment, the standard deviation is +/−0.4°2θ.

In a further alternative embodiment, Compound 2 is administered as the pharmaceutically acceptable di-hydrobromate salt, Compound 2-C.

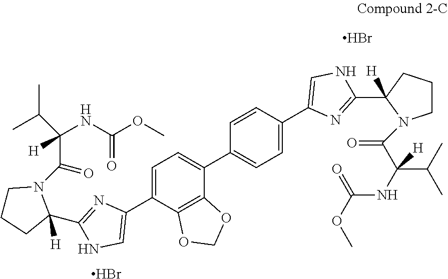 [SMILES: Br ; COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C ; Br]

The present invention also describes the morphic form of the di-hydrobromate salt of Compound 2, Compound 2-C. In one embodiment, the morphic form of Compound 2-C is characterized by an XRPD pattern that is substantially similar to that set forth in FIG. 3. In one embodiment, the morphic form of Compound 2-C is characterized by an XRPD pattern comprising at least five, at least six, at least seven, at least eight, at least nine, or at least ten 20 values from Table 4. In one embodiment, the morphic form of Compound 2-C is characterized by an XRPD pattern comprising:
a) 20 values including at least or selected from 8.5, 9.5, 14.8, 15.4, 19.0, 21.5, 22.0, 23.0, 24.2, and 30.9+/−0.2°2θ;
b) at least two, three, or four 20 values selected from 8.5, 9.5, 14.8, 15.4, 19.0, 21.5, 22.0, 23.0, 24.2, and 30.9+/−0.2°2θ;
c) at least five, six, or seven 20 values selected from 8.5, 9.5, 14.8, 15.4, 19.0, 21.5, 22.0, 23.0, 24.2, and 30.9+/−0.2°2θ;
d) at least eight or nine 20 values selected from 8.5, 9.5, 14.8, 15.4, 19.0, 21.5, 22.0, 23.0, 24.2, and 30.9+/−0.2°2θ;
e) 20 values including at least or selected from 9.5, 15.4, 21.5, 23.0, and 24.2+/−0.2°2θ; or
f) at least one 20 value selected from 9.5, 15.4, 21.5, 23.0, and 24.2+/−0.2°2θ.

In an alternative embodiment, the standard deviation is +/−0.3°2θ. In an alternative embodiment, the standard deviation is +/−0.4°2θ.

### Definitions

The term “D-configuration” as used in the context of the present invention refers to the principle configuration which mimics the natural configuration of sugar moieties as opposed to the unnatural occurring nucleosides or “L” configuration. The term “β” or “β anomer” is used with reference to nucleoside analogs in which the nucleoside base is configured (disposed) above the plane of the furanose moiety in the nucleoside analog.

The terms “coadminister” and “coadministration” or combination therapy are used to describe the administration of Compound 1 or a pharmaceutically acceptable salt thereof according to the present invention in combination with Compound 2 or a pharmaceutically acceptable salt thereof. In certain embodiments, Compound 1 or a pharmaceutically acceptable salt thereof and Compound 2 or a pharmaceutically acceptable salt thereof, for example Compound 1-A and Compound 2-A are administered with at least one other active agent, for example where appropriate at least one additional anti-HCV agent. The timing of the coadministration is best determined by the medical specialist treating the patient. It is sometimes preferred that the agents be administered at the same time or at least in a manner that allows for an overlapping pharmacologic effect of the two drugs in the treated patient. Alternatively, the drugs selected for combination therapy may be administered at different times to the patient. Of course, when more than one viral or other infection or other condition is present, the present compounds may be combined with other agents to treat that other infection or condition as required.

The term “host”, as used herein, refers to a unicellular or multicellular organism in which a HCV virus can replicate, including cell lines and animals, and typically a human. The term host specifically refers to infected cells, cells transfected with all or part of a HCV genome, and animals, in particular, primates (including chimpanzees) and humans which bear the HCV genome or a part thereof capable of treatment with the combination described herein. In most animal applications of the present invention, the host is a human patient which includes, but is not limited to a dosage regime with overlapping pharmacokinetics. Veterinary applications, in certain indications, however, are clearly anticipated by the present invention (such as chimpanzees). The host can be for example, bovine, equine, avian, canine, feline, etc., which is capable of hosting the virus.

A “pharmaceutically acceptable salt” is a derivative of the disclosed compound in which the parent compound is modified to an inorganic and organic, acid or base addition salt thereof without undue toxicity. The salts of the present compounds can be synthesized from the parent compound with a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds may optionally be provided in the form of a solvate.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids that are not unduly toxic. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The compound can be delivered in any molar ratio that delivers the desired result. For example, the compound can be provided with less than a molar equivalent of a counter ion, such as in the form of a hemi-sulfate salt. Alternatively, the compound can be provided with more than molar equivalent of counter ion, such as in the form of a di-sulfate salt. Non-limiting examples of molar ratios of the compound to the counter ion include 1:0.25, 1:0.5, 1:1, and 1:2.

### Isotopic Substitution

The present invention includes combinations of Compound 1 or a pharmaceutically acceptable salt thereof and Compound 2 or a pharmaceutically acceptable salt thereof, for example Compound 1-A and Compound 2-A, wherein one or both of the compounds has a desired isotopic substitutions of atoms at amounts above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (²H) and tritium (³H) may be used anywhere in described structures. Alternatively or in addition, isotopes of carbon, e.g., ¹³C and ¹⁴C, may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect). Achillion Pharmaceuticals, Inc. (WO/2014/169278 and WO/2014/169280) describes deuteration of nucleotides to improve their pharmacokinetic or pharmacodynamic, including at the 5-position of the molecule.

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break-down can reduce the rate of or eliminate the metabolism at that bond. At any position of the compound that a hydrogen atom may be present, the hydrogen atom can be any isotope of hydrogen, including protium (¹H), deuterium (²H) and tritium (³H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby a H-isotope, i.e., hydrogen/protium (¹H), is substituted by a H-isotope, i.e., deuterium (²H). Deuterium substitution can be partial or complete. Partial deuterium substitution means that at least one hydrogen is substituted by at least one deuterium. In certain embodiments, the isotope is 90, 95 or 99% or more enriched in an isotope at any location of interest. In some embodiments it is deuterium that is 90, 95 or 99% enriched at a desired location. Unless indicated to the contrary, the deuteration is at least 80% at the selected location. Deuteration of the nucleoside can occur at any replaceable hydrogen that provides the desired results.

### Methods of Treatment

Treatment, as used herein, refers to the administration of the combination of the present invention in an effective amount to a host, for example a human that is or may become infected with a HCV virus. In one embodiment the method of treatment comprises administration of an effective amount of Compound 1 or a pharmaceutically acceptable salt thereof and Compound 2 or a pharmaceutically acceptable salt thereof, to a host, for example a human that is or may become infected with a HCV virus. In another embodiment the method of treatment comprises administration of Compound 1-A and Compound 2 to a host, for example a human that is or may become infected with a HCV virus. In another embodiment the method of treatment comprises administration of Compound 1 and Compound 2-A to a host, for example a human that is or may become infected with a HCV virus. In another embodiment the method of treatment comprises administration of Compound 1-A and Compound 2-A to a host, for example a human that is or may become infected with a HCV virus.

The term “prophylactic” or preventative, when used, refers to the administration of a combination of the present invention to prevent or reduce the likelihood of an occurrence of the viral disorder. The present invention includes in an alternative embodiment, treatment and prophylactic or preventative therapies. In one embodiment, the combination is administered to a host who has been exposed to and thus is at risk of infection by a hepatitis C virus infection.

The invention is directed to a method of treatment of a hepatitis C virus, including drug resistant and multidrug resistant forms of HCV and related disease states, conditions, or complications of an HCV infection, including cirrhosis and related hepatotoxicities, as well as other conditions that are secondary to an HCV infection, such as weakness, loss of appetite, weight loss, breast enlargement (especially in men), rash (especially on the palms), difficulty with clotting of blood, spider-like blood vessels on the skin, confusion, coma (encephalopathy), buildup of fluid in the abdominal cavity (ascites), esophageal varices, portal hypertension, kidney failure, enlarged spleen, decrease in blood cells, anemia, thrombocytopenia, jaundice, and hepatocellular cancer, among others. The method comprises administering to a host in need thereof, typically a human, an effective amount of the combination described herein, optionally in combination with at least one additional bioactive agent, for example, an additional anti-HCV agent, further optionally in combination with a pharmaceutically acceptable carrier additive and/or excipient. In another embodiment the method comprises administering to a patient at risk of an HCV infection, an effective amount of a combination of the present invention. In another embodiment the combination as described above is used with a pharmaceutically acceptable carrier, additive, or excipient, optionally in combination with a third anti-HCV agent. In another embodiment, the combination of the present invention can be administered to a patient after a hepatitis-related liver transplantation to protect the new organ.

The combination therapy and dosage forms can also be used to treat conditions related to or occurring as a result of an HCV viral exposure. For example, the active compound can be used to treat HCV antibody-positive and HCV antigen-positive conditions, viral-based chronic liver inflammation, liver cancer resulting from advanced hepatitis C (e.g., hepatocellular carcinoma), cirrhosis, acute hepatitis C, fulminant hepatitis C, chronic persistent hepatitis C, and anti-HCV-based fatigue.

The combinations and pharmaceutical compositions described herein can also be used to treat related conditions such as anti-HCV antibody positive and antigen positive conditions, viral-based chronic liver inflammation, liver cancer resulting from advanced hepatitis C (hepatocellular carcinoma (HCC)), cirrhosis, chronic or acute hepatitis C, fulminant hepatitis C, chronic persistent hepatitis C and anti-HCV-based fatigue. The combination can also be used prophylactically to prevent or restrict the progression of clinical illness in individuals who are anti-HCV antibody- or antigen-positive or who have been exposed to hepatitis C.

### Pharmaceutical Compositions and Dosage Forms

Administration of Compound 1 or pharmaceutically acceptable salt thereof and Compound 2 or a pharmaceutically acceptable salt thereof may be carried out using any desired form, including but not limited to oral, topical, parenteral, intramuscular, intravenous, sub-cutaneous, transdermal (which may include a penetration enhancement agent), buccal, and suppository administration, among other routes of administration. In one embodiment, the active compound or combination of compounds are provided in solid dosage forms which are well known in the art and described further below. Enteric coated oral tablets may also be used to enhance bioavailability of the compounds for an oral route of administration. The most effective dosage form will depend upon the bioavailability/pharmacokinetic of the particular agents chosen as well as the severity of disease in the patient. Oral dosage forms are particularly preferred, because of ease of administration and prospective favorable patient compliance.

In certain embodiments, pharmaceutical compositions according to the present invention comprise an anti-HCV virus effective amount of each separately or a combined form of Compound 1 or a pharmaceutically acceptable salt thereof and Compound 2 or a pharmaceutically acceptable salt thereof as described herein, optionally in combination with a pharmaceutically acceptable carrier, additive, or excipient, further optionally in combination or alternation with at least one other active compound.

In one embodiment, the combination includes a solid dosage form of Compound 1 or a pharmaceutically acceptable salt thereof, for example, Compound 1-A, and Compound 2 or a pharmaceutically acceptable salt thereof, for example, Compound 2-A, in a pharmaceutically acceptable carrier. This pharmaceutical composition may contain both Compound 1 or a pharmaceutically acceptable salt thereof and Compound 2 or a pharmaceutically acceptable salt thereof or alternatively the compounds may be in separate dosage forms that are administered in a manner that the host receives the benefit of both compounds in a concerted fashion as measured by standard pharmacokinetics.

One of ordinary skill in the art will recognize that a therapeutically effective amount will vary with the infection or condition to be treated, its severity, the treatment regimen to be employed, the pharmacokinetic of the agent used, as well as the patient or subject (animal or human) to be treated, and such therapeutic amount can be determined by the attending physician or specialist.

Compound 1 or a pharmaceutically acceptable salt thereof and Compound 2 or a pharmaceutically acceptable salt thereof, for example Compound 1-A and Compound 2-A, can be formulated as one or more mixtures with one or more pharmaceutically acceptable carriers. In general, it is preferable to administer the one or more pharmaceutical composition in orally-administrable form, and in particular, one or more solid dosage forms such as a pill or tablet. Certain formulations may be administered via a parenteral, intravenous, intramuscular, topical, transdermal, buccal, subcutaneous, suppository, or other route, including intranasal spray. Intravenous and intramuscular formulations are often administered in sterile saline. One of ordinary skill in the art may modify the formulations to render them more soluble in water or another vehicle, for example, this can be easily accomplished by minor modifications (salt formulation, esterification, etc.) that are well within the ordinary skill in the art. It is also well within the routineers' skill to modify the route of administration and dosage regimen of Compound 1 or a pharmaceutically acceptable salt thereof and Compound 2 or a pharmaceutically acceptable salt thereof, for example Compound 1-A and Compound 2-A, in order to manage the pharmacokinetic of the present compounds for maximum beneficial effect in patients.

In certain pharmaceutical dosage forms, the prodrug form of the compounds, including acylated (acetylated or other), and ether (alkyl and related) derivatives, phosphate esters, thiophosphoramidates, phosphoramidates, and various salt forms of the present compounds, may be used to achieve the desired effect. One of ordinary skill in the art will recognize how to readily modify the present compounds to prodrug forms to facilitate delivery of active compounds to a targeted site within the host organism or patient. The person of ordinary skill in the art also will take advantage of favorable pharmacokinetic parameters of the prodrug forms, where applicable, in delivering the present compounds to a targeted site within the host organism or patient to maximize the intended effect of the compound.

Amounts mentioned in this disclosure typically refer to the free form (i.e., non-salt, hydrate or solvate form). The typically values described herein represent free-form equivalents, i.e., quantities as if the free form would be administered. If salts are administered the amounts need to be calculated in function of the molecular weight ratio between the salt and the free form.

The amount of Compound 1 or a pharmaceutically acceptable salt thereof and Compound 2 or a pharmaceutically acceptable salt thereof, for example Compound 1-A and Compound 2-A, included within the therapeutically active formulation according to the present invention is an effective amount to achieve the desired outcome according to the present invention, for example, for treating the HCV infection, reducing the likelihood of a HCV infection or the inhibition, reduction, and/or abolition of HCV or its secondary effects, including disease states, conditions, and/or complications which occur secondary to HCV. In general, a therapeutically effective amount of the present compounds in a pharmaceutical dosage form may range, for example, from about 0.001 mg/kg to about 100 mg/kg per day or more. Compound 1 or Compound 1-A may for example be administered in amounts ranging from about 0.1 mg/kg to about 15 mg/kg per day of the patient, depending upon the pharmacokinetics of the agent in the patient.

In certain embodiments, the pharmaceutical composition is in a dosage form that contains from about 1 mg to about 2000 mg, from about 10 mg to about 1000 mg, from about 100 mg to about 800 mg, from about 200 mg to about 600 mg, from about 300 mg to about 500 mg, or from about 400 mg to about 450 mg of Compound 1 or an equivalent amount of Compound 1-A in a unit dosage form in addition to from about 1 mg to about 2000 mg, from about 10 mg to about 1000 mg, from about 100 mg to about 800 mg of Compound 2 or an equivalent amount of Compound 2-A.

In certain embodiments, the pharmaceutical composition is in a dosage form, for example in a solid dosage form, that contains up to about 10, about 50, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, or about 1000 mg or more of Compound 1 or an equivalent amount of Compound 1-A in a unit dosage form.

In certain embodiments, the pharmaceutical composition is in a dosage form, for example in a solid dosage form, that contains up to about 10, about 50, about 60 mg, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, or about 1000 mg or more of Compound 2 or an equivalent amount of Compound 2-A in a unit dosage form.

In one embodiment, a solid dosage form containing up to about 800 mg, up to about 700 mg, up to about 600 mg, up to about 500 mg, up to about 400 mg, up to about 300 mg, up to about 200 mg, or up to about 100 mg of Compound 1 or an equivalent amount of Compound 1-A and up to about 145 mg, up to about 130 mg, up to about 125 mg, up to about 110 mg, up to about 100 mg, up to about 90 mg, up to about 75 mg, up to about 70 mg, up to about 65 mg, up to about 60 mg, up to about 55 mg, up to about 50 mg, up to about 45 mg, up to about 40 mg, up to about 35 mg, up to about 30 mg, up to about 25 mg, up to about 20 mg, up to about 15 mg, up to about 10 mg, or up to about 5 mg of Compound 2 or an equivalent amount of Compound 2-A is administered once a day to a host in need thereof for the treatment of HCV.

In one embodiment, a solid dosage form containing at least about 100 mg, at least about 200 mg, at least about 300 mg, at least about 400 mg, at least about 500 mg, at least about 600 mg, or at least about 700 mg of Compound 1 or an equivalent amount of Compound 1-A and at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 90 mg, at least about 100 mg, at least about 110 mg, at least about 125 mg, at least about 130 mg, or at least about 145 mg of Compound 2 or an equivalent amount of Compound 2-A is administered once a day to a host in need thereof for the treatment of HCV.

In one embodiment, the combination of compounds as described herein are administered as a single tablet that contains up to about 600 mg of Compound 1-A and up to about 30 mg of Compound 2 or an equivalent amount of Compound 2-A. In one embodiment, up to about 30 mg of Compound 2-A is administered.

In one embodiment, the combination of compounds as described herein are administered as a single tablet that contains up to about 600 mg of Compound 1-A and up to about 45 mg of Compound 2 or an equivalent amount of Compound 2-A. In one embodiment, up to about 45 mg of Compound 2-A is administered.

In one embodiment, the combination of compounds as described herein are administered as a single tablet that contains up to about 600 mg of Compound 1-A and up to about 60 mg of Compound 2 or an equivalent amount of Compound 2-A. In one embodiment, up to about 67 mg of Compound 2-A is administered.

In one embodiment, the combination of compounds as described herein are administered as a single tablet that contains up to about 600 mg of Compound 1-A and up to about 100 mg of Compound 2 or an equivalent amount of Compound 2-A. In one embodiment, up to about 113 mg of Compound 2-A is administered.

Alternatively, the solid dosage form of Compound 1-A or an equivalent amount of Compound 1 may be dosed in combination with a separate solid dosage form containing Compound 2 or an equivalent amount of Compound 2-A. This combination may be administered once, twice, three, or up to four times a day according to the direction of the healthcare provider. In one embodiment Compound 1-A or Compound 1 is administered in a separate schedule than Compound 2 or an equivalent amount of Compound 2-A. For example, Compound 1 or an equivalent amount of Compound 1-A may be administered twice a day while Compound 2 or an equivalent amount of Compound 2-A is only administered once a day, or vice versa: Compound 2 or an equivalent amount of Compound 2-A may be administered multiple times a day while Compound 1 or an equivalent amount of Compound 1-A is only administered once a day.

In one embodiment, a solid dosage form containing up to about 800 mg, up to about 700 mg, up to about 600 mg, up to about 500 mg, up to about 400 mg, up to about 300 mg, up to about 200 mg, or up to about 100 mg of Compound 1 or an equivalent amount of Compound 1-A is administered once a day and a separate solid dosage form containing up to about 145 mg, up to about 130 mg, up to about 125 mg, up to about 110 mg, up to about 100 mg, up to about 90 mg, up to about 75 mg, up to about 70 mg, up to about 65 mg, up to about 60 mg, up to about 55 mg, up to about 50 mg, up to about 45 mg, up to about 40 mg, up to about 35 mg, up to about 30 mg, up to about 25 mg, up to about 20 mg, up to about 15 mg, up to about 10 mg, or up to about 5 mg of Compound 2 or an equivalent amount of Compound 2-A is administered once a day to a host in need thereof for the treatment of HCV.

In one embodiment, a solid dosage form containing at least about 100 mg, at least about 200 mg, at least about 300 mg, at least about 400 mg, at least about 500 mg, at least about 600 mg, at least about 700 mg, or at least about 800 mg of Compound 1 or an equivalent amount of Compound 1-A is administered once a day and a separate solid dosage form containing at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 90 mg, at least about 100 mg, at least about 110 mg, at least about 125 mg, at least about 130 mg, or at least about 145 mg of Compound 2 or an equivalent amount of Compound 2-A is administered once a day to a host in need thereof for the treatment of HCV.

In one embodiment, a solid dosage form containing up to about 600 mg of Compound 1-A is administered once a day and a separate solid dosage form containing up to about 30 mg of Compound 2 or an equivalent amount of Compound 2-A is administered once a day to a host in need thereof for the treatment of HCV. In one embodiment, up to about 30 mg of Compound 2-A is administered.

In one embodiment, a solid dosage form containing up to about 600 mg of Compound 1-A is administered once a day and a separate solid dosage form containing up to about 45 mg of Compound 2 or an equivalent amount of Compound 2-A is administered once a day to a host in need thereof for the treatment of HCV. In one embodiment, up to about 45 mg of Compound 2-A is administered.

In one embodiment, a solid dosage form containing up to about 600 mg of Compound 1-A is administered once a day and a separate solid dosage form containing up to about 60 mg of Compound 2 or an equivalent amount of Compound 2-A is administered once a day to a host in need thereof for the treatment of HCV. In one embodiment, up to about 67 mg of Compound 2-A is administered.

In one embodiment, a solid dosage form containing up to about 600 mg of Compound 1-A is administered once a day and a separate solid dosage form containing up to about 100 mg of Compound 2 or an equivalent amount of Compound 2-A is administered once a day to a host in need thereof for the treatment of HCV. In one embodiment, up to about 113 mg of Compound 2-A is administered.

The compounds of the present combination are often administered orally, but may be administered parenterally, topically, or in suppository form, as well as intranasally, as a nasal spray or as otherwise described herein. More generally, these compounds can be administered in one or more tablets, capsules, injections, intravenous formulations, suspensions, liquids, emulsions, implants, particles, spheres, creams, ointments, suppositories, inhalable forms, transdermal forms, buccal, sublingual, topical, gel, mucosal, and the like.

In certain embodiments, the combination is administered at least once a day for up to 24 weeks. In certain embodiments, the combination is administered at least once a day for up to 12 weeks. In certain embodiments, the combination is administered at least once a day for up to 10 weeks. In certain embodiments, the combination is administered at least once a day for up to 8 weeks. In certain embodiments, the combination is administered at least once a day for up to 6 weeks. In certain embodiments, the combination is administered at least once a day for up to 4 weeks. In certain embodiments, the combination is administered at least once a day for at least 4 weeks. In certain embodiments, the combination is administered at least once a day for at least 6 weeks. In certain embodiments, the combination is administered at least once a day for at least 8 weeks. In certain embodiments, the combination is administered at least once a day for at least 10 weeks. In certain embodiments, the combination is administered at least once a day for at least 12 weeks. In certain embodiments, the combination is administered at least once a day for at least 24 weeks. In certain embodiments, the combination is administered at least every other day for up to 24 weeks, 12 weeks, up to 10 weeks, up to 8 weeks, up to 6 weeks, or up to 4 weeks. In certain embodiments, the combination is administered at least every other day for at least 4 weeks, at least 6 weeks, at least 8 weeks, at least 10 weeks, at least 12 weeks, or at least 24 weeks.

For purposes of the present invention, a prophylactically or preventive effective amount of the compositions according to the present invention falls within the same concentration range as set forth above for therapeutically effective amount and is usually the same as a therapeutically effective amount.

To prepare the pharmaceutical compositions according to the present invention, a therapeutically effective amount of Compound 1 or a pharmaceutically acceptable salt thereof and Compound 2 or pharmaceutically acceptable salts thereof, for example Compound 1-A and Compound 2-A may be intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs, and solutions, suitable carriers and additives including water, glycols, oils, alcohols, flavoring agents, preservatives, coloring agents, and the like may be used. For solid oral preparations such as powders, tablets, capsules, and for solid preparations such as suppositories, suitable carriers and additives including starches, sugar carriers, such as dextrose, manifold, lactose, and related carriers, diluents, granulating agents, lubricants, binders, disintegrating agents, and the like may be used. If desired, the tablets or capsules may be enteric-coated or sustained release by standard techniques. The use of these dosage forms may significantly enhance the bioavailability of the compounds in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents, and the like may be employed.

Liposomal suspensions (including liposomes targeted to viral antigens) may also be prepared by conventional methods to produce pharmaceutically acceptable carriers. This may be appropriate for the delivery of free nucleosides, acyl/alkyl nucleosides or phosphate ester pro-drug forms of the nucleoside compounds according to the present invention.

In typical embodiments according to the present invention, the pharmaceutical composition is used to treat, prevent, or delay a HCV infection or a secondary disease state, condition or complication of HCV.

### Solid Dosage Forms

An aspect of the invention is a fixed dosage form of the active compounds or their pharmaceutically acceptable salts thereof, optionally in a combined fixed-dosage form.

In one embodiment, the fixed dose combination includes a spray dried solid dispersion of at least one of the Compounds or its pharmaceutically acceptable salt and the composition is suitable for oral delivery. In one aspect of this embodiment, the fixed dose combination includes Compound 1 or a pharmaceutically acceptable salt thereof and Compound 2 or a pharmaceutically acceptable salt thereof, wherein at least one the Compounds is in a spray dried solid dispersion.

In another embodiment, the fixed dose combination is a granulo layered solid dispersion of at least one of the Compounds or its pharmaceutically acceptable salt and the composition is suitable for oral delivery. In one aspect of this embodiment, the fixed dose combination is a granulo layered solid dispersion that includes Compound 1 or a pharmaceutically acceptable salt thereof and Compound 2 or a pharmaceutically acceptable salt thereof. In certain embodiments, a spray dried dispersion or granulo layered solid dispersion component is prepared using crystalline Compound 1-A. In certain embodiments, a spray dried dispersion or granulo layered solid dispersion component is prepared using crystalline Compound 2-A. In an alternative embodiment, Compound 1 or a pharmaceutically acceptable salt, for example, Compound 1-A, or Compound 2 or a pharmaceutically acceptable, for example Compound 2-A, can be delivered as an amorphous compound.

In other embodiments, the solid dispersion also contains at least one excipient selected from copovidone, poloxamer and HPMC-AS. In one embodiment the poloxamer is Poloxamer 407 or a mixture of poloxamers that may include Poloxamer 407. In one embodiment HPMC-AS is HPMC-AS-L.

In other embodiments, a fixed dose composition prepared from Compound 1 or a pharmaceutically acceptable salt thereof and Compound 2 or a pharmaceutically acceptable salt thereof, also comprises one or more of the following excipients: a phosphoglyceride; phosphatidylcholine; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohol such as polyethylene glycol (PEG); polyoxyethylene-9-lauryl ether; a surface active fatty acid, such as palmitic acid or oleic acid; fatty acid; fatty acid monoglyceride; fatty acid diglyceride; fatty acid amide; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebroside; dicetylphosphate; dipalmitoylphosphatidylglycerol; stearylamine; dodecylamine; hexadecyl-amine; acetyl palmitate; glycerol ricinoleate; hexadecyl stearate; isopropyl myristate; tyloxapol; poly(ethylene glycol)5000-phosphatidylethanolamine; poly(ethylene glycol)400-monostearate; phospholipid; synthetic and/or natural detergent having high surfactant properties; deoxycholate; cyclodextrin; chaotropic salt; ion pairing agent; glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid; pullulan, cellulose, microcrystalline cellulose, silicified microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan, mannitol, sorbitol, xylitol, erythritol, maltitol, and lactitol, a pluronic polymer, polyethylene, polycarbonate (e.g., poly(1,3-dioxan-2one)), polyanhydride (e.g., poly(sebacic anhydride)), polypropylfumerate, polyamide (e.g. polycaprolactam), polyacetal, polyether, polyester (e.g., polylactide, polyglycolide, polylactide-co-glycolide, polycaprolactone, polyhydroxyacid (e.g., poly((β-hydroxyalkanoate))), poly(orthoester), polycyanoacrylate, polyvinyl alcohol, polyurethane, polyphosphazene, polyacrylate, polymethacrylate, polyurea, polystyrene, and polyamine, polylysine, polylysine-PEG copolymer, and poly(ethyleneimine), poly(ethylene imine)-PEG copolymer, glycerol monocaprylocaprate, propylene glycol, Vitamin E TPGS (also known as d-α-Tocopheryl polyethylene glycol 1000 succinate), gelatin, titanium dioxide, polyvinylpyrrolidone (PVP), hydroxypropyl methyl cellulose (HPMC), hydroxypropyl cellulose (HPC), methyl cellulose (MC), block copolymers of ethylene oxide and propylene oxide (PEO/PPO), polyethyleneglycol (PEG), sodium carboxymethylcellulose (NaCMC), or hydroxypropylmethyl cellulose acetate succinate (HPMCAS).

In other embodiments, a fixed dose composition prepared from Compound 1 or a pharmaceutically acceptable salt thereof and Compound 2 or a pharmaceutically acceptable salt thereof, also comprises one or more of the following surfactants: polyoxyethylene glycol, polyoxypropylene glycol, decyl glucoside, lauryl glucoside, octyl glucoside, polyoxyethylene glycol octylphenol, Triton X-100, glycerol alkyl ester, glyceryl laurate, cocamide MEA, cocamide DEA, dodecyldimethylamine oxide, and poloxamers. Examples of poloxamers include, poloxamers 188, 237, 338 and 407. These poloxamers are available under the trade name Pluronic® (available from BASF, Mount Olive, N.J.) and correspond to Pluronic® F-68, F-87, F-108 and F-127, respectively. Poloxamer 188 (corresponding to Pluronic® F-68) is a block copolymer with an average molecular mass of about 7,000 to about 10,000 Da, or about 8,000 to about 9,000 Da, or about 8,400 Da. Poloxamer 237 (corresponding to Pluronic® F-87) is a block copolymer with an average molecular mass of about 6,000 to about 9,000 Da, or about 6,500 to about 8,000 Da, or about 7,700 Da. Poloxamer 338 (corresponding to Pluronic® F-108) is a block copolymer with an average molecular mass of about 12,000 to about 18,000 Da, or about 13,000 to about 15,000 Da, or about 14,600 Da. Poloxamer 407 (corresponding to Pluronic® F-127) is a polyoxyethylene-polyoxypropylene triblock copolymer in a ratio of between about E101 P56 E101 to about E106 P70 E106, or about E101 P56E101, or about E106 P70 E106, with an average molecular mass of about 10,000 to about 15,000 Da, or about 12,000 to about 14,000 Da, or about 12,000 to about 13,000 Da, or about 12,600 Da.

In yet other embodiments, a fixed dose composition prepared from Compound 1 or a pharmaceutically acceptable salt thereof and Compound 2 or a pharmaceutically acceptable salt thereof, also comprises one or more of the following surfactants: polyvinyl acetate, cholic acid sodium salt, dioctyl sulfosuccinate sodium, hexadecyltrimethyl ammonium bromide, saponin, sugar esters, Triton X series, sorbitan trioleate, sorbitan mono-oleate, polyoxyethylene (20) sorbitan monolaurate, polyoxyethylene (20) sorbitan monooleate, oleyl polyoxyethylene (2) ether, stearyl polyoxyethylene (2) ether, lauryl polyoxyethylene (4) ether, block copolymers of oxyethylene and oxypropylene, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, cetylpyridinium chloride, benzalkonium chloride, olive oil, glyceryl monolaurate, corn oil, cotton seed oil, and sunflower seed oil.

In alternative embodiments, a fixed dose composition prepared from Compound 1 or a pharmaceutically acceptable salt thereof and Compound 2 or a pharmaceutically acceptable salt thereof, is prepared by a process that includes solvent or dry granulation optionally followed by compression or compaction, spray drying, nano-suspension processing, hot melt extrusion, extrusion/spheronization, molding, spheronization, layering (e.g., spray layering suspension or solution), or the like. Examples of such techniques include direct compression, using appropriate punches and dies, for example wherein the punches and dies are fitted to a suitable tableting press; wet granulation using suitable granulating equipment such as a high shear granulator to form wetted particles to be dried into granules; granulation followed by compression using appropriate punches and dies, wherein the punches and dies are fitted to a suitable tableting press; extrusion of a wet mass to form a cylindrical extrudate to be cut into desire lengths or break into lengths under gravity and attrition; extrusion/spheronization where the extrudate is rounded into spherical particles and densified by spheronization; spray layering of a suspension or solution onto an inert core using a technique such as a convention pan or Wurster column; injection or compression molding using suitable molds fitted to a compression unit; and the like.

Exemplary disintegrants include alginic acid, carboxymethylcellulose calcium, carboxymethylcellulose sodium, cross-linked sodium carboxymethylcellulose (sodium croscarmellose), powdered cellulose, chitosan, croscarmellose sodium, crospovidone, guar gum, low substituted hydroxypropyl cellulose, methyl cellulose, microcrystalline cellulose, sodium alginate, sodium starch glycolate, partially pregelatinized starch, pregelatinized starch, starch, sodium carboxymethyl starch, and the like, or a combination thereof.

Exemplary lubricants include calcium stearate, magnesium stearate, glyceryl behenate, glyceryl palmitostearate, hydrogenated castor oil, light mineral oil, sodium lauryl sulfate, magnesium lauryl sulfate, sodium stearyl fumarate, stearic acid, zinc stearate, silicon dioxide, colloidal silicon dioxide, dimethyldichlorosilane treated with silica, talc, or a combination thereof.

The dosage form cores described herein may be coated to result in coated tablets. The dosage from cores can be coated with a functional or non-functional coating, or a combination of functional and non-functional coatings. “Functional coating” includes tablet coatings that modify the release properties of the total composition, for example, a sustained-release or delayed-release coating. “Non-functional coating” includes a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition. A non-functional coating can also mask the taste of the uncoated composition including the active pharmaceutical ingredient. A coating may comprise a light blocking material, a light absorbing material, or a light blocking material and a light absorbing material.

Exemplary polymethacrylates include copolymers of acrylic and methacrylic acid esters, such as a. an aminomethacrylate copolymer USP/NF such as a poly(butyl methacrylate, (2-dimethyl aminoethyl)methacrylate, methyl methacrylate) 1:2:1 (e.g., EUDRAGIT E 100, EUDRAGIT EPO, and EUDRAGIT E 12.5; CAS No. 24938-16-7); b. a poly(methacrylic acid, ethyl acrylate) 1:1 (e.g., EUDRAGIT L30 D-55, EUDRAGIT L100-55, EASTACRYL 30D, KOLLICOAT MAE 30D AND 30DP; CAS No. 25212-88-8); c. a poly(methacrylic acid, methyl methacrylate) 1:1 (e.g., EUDRAGIT L 100, EUDRAGIT L 12.5 and 12.5 P; also known as methacrylic acid copolymer, type A NF; CAS No. 25806-15-1); d. a poly(methacrylic acid, methyl methacrylate) 1:2 (e.g., EUDRAGIT S 100, EUDRAGIT S 12.5 and 12.5P; CAS No. 25086-15-1); e. a poly(methyl acrylate, methyl methacrylate, methacrylic acid) 7:3:1 (e.g., Eudragit FS 30 D; CAS No. 26936-24-3); f. a poly(ethyl acrylate, methylmethacrylate, trimethylammonioethyl methacrylate chloride) 1:2:0.2 or 1:2:0.1 (e.g., EUDRAGITS RL 100, RL PO, RL 30 D, RL 12.5, RS 100, RS PO, RS 30 D, or RS 12.5; CAS No. 33434-24-1); g. a poly(ethyl acrylate, methyl methacrylate) 2:1 (e.g., EUDRAGIT NE 30 D, Eudragit NE 40D, Eudragit NM 30D; CAS No. 9010-88-2); and the like, or a combination thereof.

Suitable alkylcelluloses include, for example, methylcellulose, ethylcellulose, and the like, or a combination thereof. Exemplary water based ethylcellulose coatings include AQUACOAT, a 30% dispersion further containing sodium lauryl sulfate and cetyl alcohol, available from FMC, Philadelphia, Pa.; SURELEASE a 25% dispersion further containing a stabilizer or other coating component (e.g., ammonium oleate, dibutyl sebacate, colloidal anhydrous silica, medium chain triglycerides, etc.) available from Colorcon, West Point, Pa.; ethyl cellulose available from Aqualon or Dow Chemical Co (Ethocel), Midland, Mich. Those skilled in the art will appreciate that other cellulosic polymers, including other alkyl cellulosic polymers, can be substituted for part or all of the ethylcellulose.

Other suitable materials that can be used to prepare a functional coating include hydroxypropyl methylcellulose acetate succinate (HPMCAS); cellulose acetate phthalate (CAP); a polyvinylacetate phthalate; neutral or synthetic waxes, fatty alcohols (such as lauryl, myristyl, stearyl, cetyl or specifically cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, or a combination thereof. Suitable waxes include beeswax, glycowax, castor wax, carnauba wax, microcrystalline wax, candelilla, and wax-like substances, e.g., material normally solid at room temperature and having a melting point of from about 30° C. to about 100° C., or a combination thereof.

In other embodiments, a functional coating may include digestible, long chain (e.g., C8-C50, specifically C12-C40), substituted or unsubstituted hydrocarbons, such as fatty acids, fatty alcohols, glyceryl esters of fatty acids, mineral and vegetable oils, waxes, or a combination thereof. Hydrocarbons having a melting point of between about 25° C. and about 90° C. may be used. Specifically, long chain hydrocarbon materials, fatty (aliphatic) alcohols can be used.

The coatings can optionally contain additional pharmaceutically acceptable excipients such as a plasticizer, a stabilizer, a water-soluble component (e.g., pore formers), an anti-tacking agent (e.g., talc), a surfactant, and the like, or a combination thereof.

A functional coating may include a release-modifying agent, which affects the release properties of the functional coating. The release-modifying agent can, for example, function as a pore-former or a matrix disrupter. The release-modifying agent can be organic or inorganic, and include materials that can be dissolved, extracted or leached from the coating in the environment of use. The release-modifying agent can comprise one or more hydrophilic polymers including cellulose ethers and other cellulosics, such as hydroxypropyl methylcellulose, hydroxypropylcellulose, hydroxyethylcellulose, methyl cellulose, cellulose acetate phthalate, or hydroxypropyl methylcellulose acetate phthalate; povidone; polyvinyl alcohol; an acrylic polymer, such as gastric soluble Eudragit FS 30D, pH sensitive Eudragit L30D 55, L 100, S 100, or L 100-55; or a combination thereof. Other exemplary release-modifying agents include a povidone; a saccharide (e.g., lactose, and the like); a metal stearate; an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); or a combination thereof. Exemplary matrix disrupters include water insoluble organic or inorganic material. Organic polymers including but not limited to cellulose, cellulose ethers such as ethylcellulose, cellulose esters such as cellulose acetate, cellulose acetate butyrate and cellulose acetate propionate; and starch can function as matrix disrupters. Examples or inorganic disrupters include many calcium salts such as mono-, di- and tri calcium phosphate; silica and, talc.

The coating may optionally contain a plasticizer to improve the physical properties of the coating. For example, because ethylcellulose has a relatively high glass transition temperature and does not form flexible films under normal coating conditions, it may be advantageous to add plasticizer to the ethylcellulose before using the same as a coating material. Generally, the amount of plasticizer included in a coating solution is based on the concentration of the polymer, e.g., can be from about 1% to about 200% depending on the polymer but is most often from about 1 wt % to about 100 wt % of the polymer. Concentrations of the plasticizer, however, can be determined by routine experimentation.

Examples of plasticizers for ethylcellulose and other celluloses include plasticizers such as dibutyl sebacate, diethyl phthalate, triethyl citrate, tributyl citrate, triacetin, or a combination thereof, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, or a combination thereof, although it is possible that other plasticizers (such as acetylated monoglycerides, phthalate esters, castor oil, etc.) can be used.

Suitable methods can be used to apply the coating material to the surface of the dosage form cores. Processes such as simple or complex coacervation, interfacial polymerization, liquid drying, thermal and ionic gelation, spray drying, spray chilling, fluidized bed coating, pan coating, or electrostatic deposition may be used.

In certain embodiments, an optional intermediate coating is used between the dosage form core and an exterior coating. Such an intermediate coating can be used to protect the active agent or other component of the core subunit from the material used in the exterior coating or to provide other properties. Exemplary intermediate coatings typically include water-soluble film forming polymers. Such intermediate coatings may include film forming polymers such as hydroxyethyl cellulose, hydroxypropyl cellulose, gelatin, hydroxypropyl methylcellulose, polyethylene glycol, polyethylene oxide, and the like, or a combination thereof, and a plasticizer. Plasticizers can be used to reduce brittleness and increase tensile strength and elasticity. Exemplary plasticizers include polyethylene glycol propylene glycol and glycerin.

### Combination and Alternation Therapy

Drug resistance sometimes occurs by mutation of a gene that encodes for an enzyme used in viral replication. The efficacy of a combination therapy against an HCV infection, can be prolonged, augmented, or restored by adding an additional compound to the combination therapy. This further combined therapy may be administered together or in alternation with another, and perhaps even two or three other, antiviral compounds that induce a different mutation or act through a different pathway, from that of the principle combination. Alternatively, the pharmacokinetic, bio-distribution, half-life, or other parameter of the combination can be altered by such combination therapy (which may include alternation therapy if considered concerted).

This invention already provides an advantageous combination therapy for the treatment of HCV, or a disorder associated with an HCV infection, by administering a selected NS5B inhibitor with an NS5A inhibitor. Additional therapeutic effects may be achieved by adding a third, fourth, or even fifth active agent either co-formulated or provided separately.

Since Compound 1 and Compound 1-A are NS5B polymerase inhibitors and Compound 2 and Compound 2-A are NS5A inhibitors it may be useful to administer Compound 1 and Compound 2 to a host in combination with, for example a
(1) Protease inhibitor, such as an NS3/4A protease inhibitor;
(2) Another NS5A inhibitor;
(3) Another NS5B polymerase inhibitor;
(4) NS5B non-substrate inhibitor;
(5) Interferon alfa-2a, which may be pegylated or otherwise modified, and/or ribavirin;
(6) Non-substrate-based inhibitor;
(7) Helicase inhibitor;
(8) Antisense oligodeoxynucleotide (S-ODN);
(9) Aptamer;
(10) Nuclease-resistant ribozyme;
(11) iRNA, including microRNA and SiRNA;
(12) Antibody, partial antibody or domain antibody to the virus, or
(13) Viral antigen or partial antigen that induces a host antibody response.

Non limiting examples of additional anti-HCV agents that can be administered in further combination or alternation with the combination of the present invention, include
(i) protease inhibitors such as telaprevir (Incivek®), boceprevir (Victrelis™), simeprevir (Olysio™), paritaprevir (ABT-450), glecaprevir (ABT-493), ritonavir (Norvir), ACH-2684, AZD-7295, BMS-791325, danoprevir, Filibuvir, GS-9256, GS-9451, MK-5172, Setrobuvir, Sovaprevir, Tegobuvir, VX-135, VX-222, ALS-220, and voxilaprevir.
(ii) NS5A inhibitor such as ACH-2928, ACH-3102, IDX-719, daclatasvir, ledispasvir, velpatasvir (Epclusa), elbasvir (MK-8742), grazoprevir (MK-5172), and Ombitasvir (ABT-267);
(iii) NS5B inhibitors such as AZD-7295, Clemizole, dasabuvir (Exviera), ITX-5061, PPI-461, PPI-688, sofosbuvir (Sovaldi®), MK-3682, and mericitabine;
(iv) NS5B inhibitors such as ABT-333, and MBX-700;
(v) Antibody such as GS-6624;
(vi) Combination drugs such as Harvoni (ledipasvir/sofosbuvir); Viekira Pak (ombitasvir/paritaprevir/ritonavir/dasabuvir); Viekirax (ombitasvir/paritaprevir/ritonavir); G/P (paritaprevir and glecaprevir); Technivie™ (ombitasvir/paritaprevir/ritonavir), Epclusa (sofosbuvir/velpatasvir), Zepatier (elbasvir and grazoprevir), Mavyret (glecaprevir and pibrentasvir), and Vosevi (Sofosbuvir, velpatasvir, and voxilaprevir).

If the combination is administered to treat advanced hepatitis C virus leading to liver cancer or cirrhosis, in one embodiment, the compound can be administered in combination or alternation with another drug that is typically used to treat hepatocellular carcinoma (HCC), for example, as described by Andrew Zhu in “New Agents on the Horizon in Hepatocellular Carcinoma” Therapeutic Advances in Medical Oncology, V 5(1), January 2013, 41-50. Examples of suitable compounds for combination therapy where the host has or is at risk of HCC include anti-angiogenic agents, sunitinib, brivanib, linifanib, ramucirumab, bevacizumab, cediranib, pazopanib, TSU-68, lenvatinib, antibodies against EGFR, mTor inhibitors, MEK inhibitors, and histone decetylace inhibitors, capecitabine, cisplatin, carboplatin, doxorubicin, 5-fluorouracil, gemcitabine, irinotecan, oxaliplatin, topotecan, and other topoisomerases.

### EXAMPLES

### General Methods

¹H, ¹⁹F and ³¹P NMR spectra were recorded on a 400 MHz Fourier transform Brucker spectrometer. Spectra were obtained DMSO-d₆ unless stated otherwise. The spin multiplicities are indicated by the symbols s (singlet), d (doublet), t (triplet), m (multiplet) and, br (broad). Coupling constants (J) are reported in Hz. The reactions were generally carried out under a dry nitrogen atmosphere using Sigma-Aldrich anhydrous solvents. All common chemicals were purchased from commercial sources.

The following abbreviations are used in the Examples:

DCM: Dichloromethane

EtOAc: Ethyl acetate

EtOH: Ethanol

GT: Genotype

HPLC: High pressure liquid chromatography

NaOH: Sodium hydroxide

Na₂SO₄: Sodium sulphate (anhydrous)

MeOH: Methanol

Na₂SO₄: Sodium sulfate

NH₄Cl: Ammonium chloride

PE: Petroleum ether

Silica gel (230 to 400 mesh, Sorbent)

t-BuMgCl: t-Butyl magnesium chloride

THF: Tetrahydrofuran (THF), anhydrous

TP: Triphosphate

### Example 1. Synthesis of Compound 1

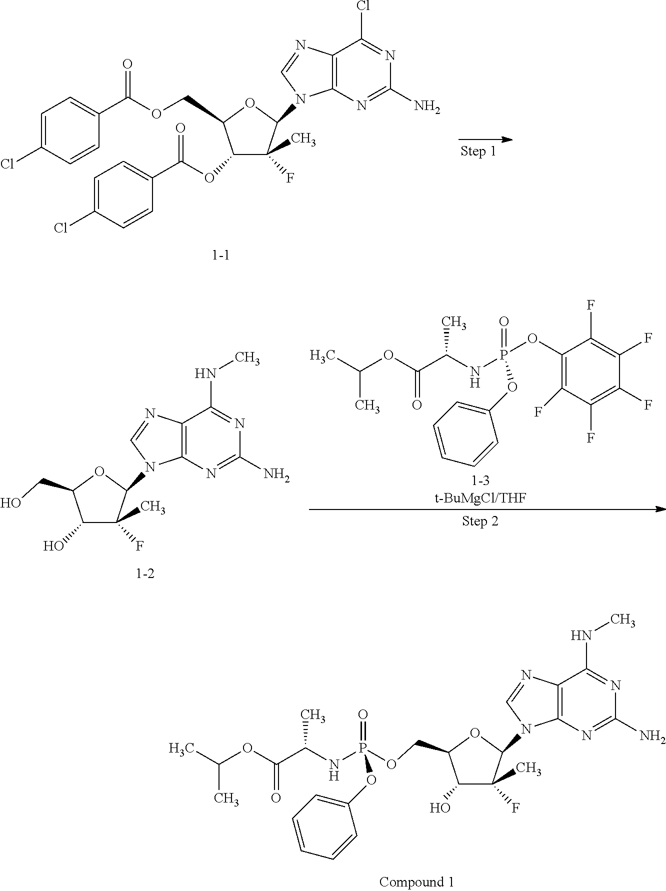 [SMILES: C[C@@]1(F)[C@H](OC(=O)c2ccc(Cl)cc2)[C@@H](COC(=O)c2ccc(Cl)cc2)O[C@H]1n1cnc2c(Cl)nc(N)nc21 ; CNc1nc(N)nc2c1ncn2[C@@H]1O[C@H](CO)[C@@H](O)[C@@]1(C)F ; CC(C)OC(=O)[C@H](C)N[P@](=O)(Oc1ccccc1)Oc1c(F)c(F)c(F)c(F)c1F ; CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

Step 1: Synthesis of (2R,3R,4R,5R)-5-(2-Amino-6-(methylamino)-9H-purin-9-yl)-4-fluoro-2-(hydroxymethyl)-4-methyltetrahydrofuran-3-ol (1-2) A 50 L flask was charged with methanol (30 L) and stirred at 10±5° C. NH₂CH₃ (3.95 Kg) was slowly ventilated into the reactor at 10±5° C. Compound 1-1 (3.77 kg) was added in batches at 20±5° C. and stirred for 1 hour to obtain a clear solution. The reaction was stirred for an additional 6-8 hours, at which point HPLC indicated that the intermediate was less than 0.1% of the solution. The reactor was charged with solid NaOH (254 g), stirred for 30 minutes and concentrated at 50±5° C. (vacuum degree: −0.095). The resulting residue was charged with EtOH (40 L) and re-slurried for 1 hour at 60° C. The mixture was then filtered through celite and the filter cake was re-slurried with EtOH (15 L) for 1 hour at 60° C. The filtrate was filtered once more, combined with the filtrate from the previous filtration, and then concentrated at 50±5° C. (vacuum degree: −0.095). A large amount of solid was precipitated. EtOAc (6 L) was added to the solid residue and the mixture was concentrated at 50±5° C. (vacuum degree: −0.095). DCM was then added to the residue and the mixture was re-slurried at reflux for 1 hour, cooled to room temperature, filtered, and dried at 50±5° C. in a vacuum oven to afford compound 1-2 as an off-white solid (1.89 Kg, 95.3%, purity of 99.2%).

### Step 2: Synthesis of isopropyl((S)-(((2R,3R,4R,5R)-5-(2-Amino-6-(methylamino)-9H-purin-9-yl)-4-fluoro-3-hydroxy-4-methyltetrahydrofuran-2-yl)methoxy)(phenoxy)phosphoryl)-L-alaninate (Compound 1)

Compound 1-2 and compound 1-3 (isopropyl ((perfluorophenoxy)(phenoxy)phosphoryl)-L-alaninate) were dissolved in THF (1 L) and stirred under nitrogen. The suspension was then cooled to a temperature below −5° C. and a 1.7 M solution of t-BuMgCl solution (384 mL) was slowly added over 1.5 hours while a temperature of 5-10° C. was maintained. A solution of NH₄C₁ (2 L) and water (8 L) was added to the suspension at room temperature followed by DCM. The mixture was stirred for 5 minutes before a 5% aqueous solution of K₂CO₃ (10 L) was added and the mixture was stirred for 5 additional minutes before filtering through diatomite (500 g). The diatomite was washed with DCM and the filtrate was separated. The organic phase was washed with a 5% aqueous K₂CO₃ solution (10 L×2), brine (10 L×3), and dried over Na₂SO₄ (500 g) for approximately 1 hour. Meanwhile, this entire process was repeated 7 times in parallel and the 8 batches were combined. The organic phases were filtered and concentrated at 45±5° C. (vacuum degree of 0.09 Mpa). EtOAc was added and the mixture was stirred for 1 hour at 60° C. and then at room temperature for 18 hours. The mixture was then filtered and washed with EtOAc (2 L) to afford crude Compound 1. The crude material was dissolved in DCM (12 L), heptane (18 L) was added at 10-20° C., and the mixture was allowed to stir for 30 minutes at this temperature. The mixture was filtered, washed with heptane (5 L), and dried at 50±5° C. to afford pure Compound 1 (1650 g, 60%).

Amorphous Compound 1: ¹H NMR (400 MHz, DMSO-d₆) δ ppm 1.01-1.15 (m, 9H), 1.21 (d, J=7.20 Hz, 3H), 2.75-3.08 (m, 3H), 3.71-3.87 (m, 1H), 4.02-4.13 (m, 1H), 4.22-4.53 (m, 3H), 4.81 (s, 1H), 5.69-5.86 (m, 1H), 6.04 (br d, J=19.33 Hz, 4H), 7.12-7.27 (m, 3H), 7.27-7.44 (m, 3H), 7.81 (s, 1H)

Crystalline Compound 1: ¹H NMR (400 MHz, DMSO-d₆) δ ppm 0.97-1.16 (m, 16H), 1.21 (d, J=7.07 Hz, 3H), 2.87 (br s, 3H), 3.08 (s, 2H), 3.79 (br d, J=7.07 Hz, 1H), 4.08 (br d, J=7.58 Hz, 1H), 4.17-4.55 (m, 3H), 4.81 (quin, J=6.25 Hz, 1H), 5.78 (br s, 1H), 5.91-6.15 (m, 4H), 7.10-7.26 (m, 3H), 7.26-7.44 (m, 3H), 7.81 (s, 1H)

### Example 2. Synthesis of Compound 1-A

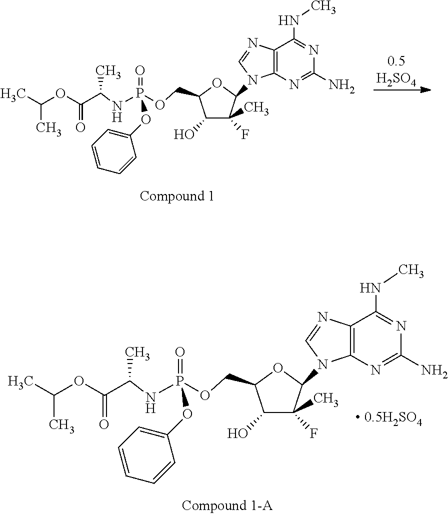 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F ; CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

A 250 mL flask was charged with MeOH (151 mL) and the solution was cooled to 0-5° C. A concentrated solution of H₂SO₄ was added dropwise over 10 minutes. A separate flask was charged with Compound 1 (151 g) and acetone (910 mL), and the H₂SO₄/MeOH solution was added dropwise at 25-30° C. over 2.5 hours. A large amount of solid was precipitated. After the solution was stirred for 12-15 hours at 25-30° C., the mixture was filtered, washed with MeOH/acetone (25 mL/150 mL), and dried at 55-60° C. in vacuum to afford Compound 1-A (121 g, 74%). ¹HNMR: (400 MHz, DMSO-d₆): δ 8.41 (br, 1H), 7.97 (s, 1H), 7.36 (t, J=8.0 Hz, 2H), 7.22 (d, J=8.0 Hz, 2H), 7.17 (t, J=8.0 Hz, 1H), 6.73 (s, 2H), 6.07 (d, J=8.0 Hz, 1H), 6.00 (dd, J=12.0, 8.0 Hz, 1H), 5.81 (br, 1H), 4.84-4.73 (m, 1H), 4.44-4.28 (m, 3H), 4.10 (t, J=8.0 Hz, 2H), 3.85-3.74 (m, 1H), 2.95 (s, 3H), 1.21 (s, J=4.0 Hz, 3H), 1.15-1.10 (m, 9H).

### Example 3. Salt Investigation of Compound 2

As shown in the Table 1, sixteen acids (four inorganic acids and twelve organic acids) were used in the salt investigation of Compound 2. The free base (0.1 g-1 g) was added to the solvent (1-10 mL), and the mixture was heated to 40-80° C. The acid was added and after stirring for 30 minutes to 1 hour, the mixture was slowly cooled to 5±5° C. After cooling, the mixture was either a clear liquid, a viscous oil, or a precipitated solid. The precipitated solid was filtered, dried under reduced pressure and characterized by XPRD.

TABLE 1
Compound 2 Salt Investigation Conditions

| | | | Crystalline or |
|---|---|---|---|
| Acid | Solvent | Result | Amorphous |
| Di•½H₂SO₄ | i-PrOH | Good solid | amorphous |
| | EtOH | Good solid | amorphous |
| | CH₃CN | Good solid | amorphous |
| | MeOH + i-PrOH | Good solid | mixed crystal |
| | MeOH + EtOAc | Good solid | mixed crystal |
| | MeOH | Good solid | crystalline |
| Di•H₂SO₄ | H₂O | No solid precipitation | / |
| | i-PrOH | Good solid | amorphous |
| | MeOH | Good solid | oil |
| | EtOH | Poor solid | / |
| | ACN | oil | / |
| H₃PO₄ | iPrOH | Precipitation | N |
| | EtOH | No solid precipitation | |
| | H₂O | No solid precipitation | |
| | MeOH + i-PrOH | Good solid | amorphous |
| | MeOH + EtOAc | Good solid | amorphous |
| | MeOH + acetone | No solid precipitation | / |
| HNO₃ | iPrOH | Good solid | amorphous |
| | EtOH | No solid precipitation | |
| | H₂O | Viscous oil | |
| | ACN | Precipitation | crystalline |
| | Acetone | Viscous oil | / |
| HBr | iPrOH | Precipitation | crystalline |
| | H2O | Viscous oil | / |
| MeSO₃H | ACN | No solid precipitation | / |
| | MTBE | solid | amorphous |
| PhSO₃H | ACN | Viscous oil | / |
| | MTBE | solid | amorphous |
| citrate | ACN | No solid precipitation | / |
| | MTBE | Viscous oil | / |
| maleate | ACN | Viscous oil | / |
| | MTBE | solid | amorphous |
| fumarate | ACN | solid | amorphous |
| | MTBE | solid | amorphous |
| succinate | ACN | Viscous oil | / |
| | MTBE | solid | amorphous |
| D-Camphorsulfonate | ACN | No solid precipitation | / |
| | MTBE | solid | amorphous |
| malate | ACN | Viscous oil | / |
| | MTBE | solid | amorphous |
| L-tartrate | ACN | solid | amorphous |
| | MTBE | solid | amorphous |
| D-tartrate | ACN | solid | amorphous |
| | MTBE | solid | amorphous |
| S-mandelate | ACN | No solid precipitation | / |
| | MTBE | solid | amorphous |
| Benzoic acid | ACN | No solid precipitation | / |
| | MTBE | solid | amorphous |

All the organic acids afforded amorphous solids or oils despite trying several alcohol solids (MeOH, EtOH, and i-PrOH). The three acids that afforded a crystalline solid were di-hemisulfate, di-HBr and di-HNO₃, but the crystalline solid for each of these acids was only obtained using a specific solvent. For example, the crystallization investigation of the di-hemisulfate salt was conducted with i-PrOH, EtOH, CH₃CN, MeOH/i-PrOH, MeOH/EtOAc, and MeOH, but only the test in the MeOH resulted in a crystalline solid. Further, only the test in CH₃CN resulted in a crystalline solid of the di-nitrate salt despite other studies in i-PrOH, EtOH, H₂O, and acetone and the di-hydrobromate salt only crystallized in the presence of i-PrOH, but not H₂O.

The di-hemisulfate crystalline solid is the preferred solid for drug development for the combination of the present invention.

The procedures and XRPD characterization for the crystalline di-hemisulfate, di-HBr, and di-HNO₃ solids of Compound 2 are below.

### Di-Hemisulfate Compound 2-A

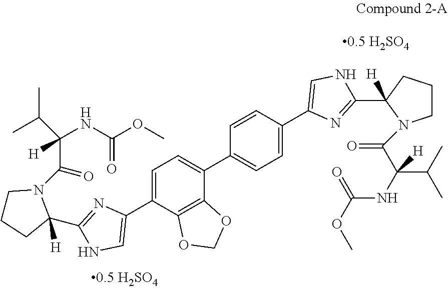 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

The free base (1 g) was dissolved in 6 mL methanol and the mixture was heated to 45±5° C. H₂SO₄ (0.125 g, 1 eq.) was added at 45±5° C. with stirring for 1 hour and the mixture was cooled to 5±5° C. The resulting solid was filtrated and dried under reduced pressure to afford 0.97 g of crystalline solid Compound 2-A (Yield: 86%). The peaks of the XRPD pattern are shown in Table 2. The XRPD pattern is shown in FIG. 1A and the DSC is shown in FIG. 1B.

TABLE 2
Compound 2-A XRPD Pattern Peaks

| Peak No. | 2-Theta | d | BG | Height | Height % | Area | Area % | FWHM |
|---|---|---|---|---|---|---|---|---|
| 1 | 5.499 | 16.0576 | 269 | 110 | 6.1 | 953 | 5.4 | 0.148 |
| 2 | 7.310 | 12.0830 | 322 | 1807 | 100.0 | 17668 | 100.0 | 0.167 |
| 3 | 7.853 | 11.2489 | 342 | 476 | 26.3 | 4704 | 26.6 | 0.168 |
| 4 | 9.066 | 9.7465 | 378 | 104 | 5.8 | 615 | 3.5 | 0.101 |
| 5 | 9.653 | 9.1551 | 372 | 96 | 5.3 | 1828 | 10.3 | 0.325 |
| 6 | 11.061 | 7.9925 | 420 | 159 | 8.8 | 1341 | 7.6 | 0.144 |
| 7 | 12.024 | 7.3544 | 434 | 761 | 42.1 | 16319 | 92.4 | 0.366 |
| 8 | 12.201 | 7.2481 | 430 | 707 | 39.1 | 12501 | 70.8 | 0.284 |
| 9 | 13.344 | 6.6299 | 421 | 125 | 6.9 | 1415 | 8.0 | 0.193 |
| 10 | 14.652 | 6.0407 | 433 | 1038 | 57.4 | 9268 | 52.5 | 0.152 |
| 11 | 15.752 | 5.6212 | 454 | 690 | 38.2 | 9199 | 52.1 | 0.227 |
| 12 | 16.135 | 5.4887 | 456 | 411 | 22.7 | 6309 | 35.7 | 0.262 |
| 13 | 16.494 | 5.3699 | 491 | 764 | 42.3 | 7587 | 42.9 | 0.169 |
| 14 | 17.218 | 5.1458 | 467 | 370 | 20.5 | 3203 | 18.1 | 0.148 |
| 15 | 18.201 | 4.8700 | 513 | 1226 | 67.8 | 12734 | 72.1 | 0.177 |
| 16 | 19.059 | 4.6526 | 530 | 406 | 22.5 | 5564 | 31.5 | 0.234 |
| 17 | 19.823 | 4.4750 | 529 | 338 | 18.7 | 8268 | 46.8 | 0.417 |
| 18 | 20.087 | 4.4168 | 527 | 245 | 13.6 | 6166 | 34.9 | 0.429 |
| 19 | 20.265 | 4.3785 | 526 | 235 | 13.0 | 4983 | 28.2 | 0.361 |
| 20 | 20.950 | 4.2367 | 521 | 61 | 3.4 | 493 | 2.8 | 0.130 |
| 21 | 22.106 | 4.0177 | 608 | 211 | 11.7 | 1561 | 8.8 | 0.126 |
| 22 | 22.406 | 3.9647 | 599 | 384 | 21.3 | 10635 | 60.2 | 0.472 |
| 23 | 22.651 | 3.9223 | 564 | 408 | 22.6 | 10801 | 61.1 | 0.451 |
| 24 | 23.191 | 3.8322 | 643 | 278 | 15.4 | 3482 | 19.7 | 0.213 |
| 25 | 24.161 | 3.6805 | 544 | 135 | 7.5 | 1510 | 8.5 | 0.191 |
| 26 | 24.616 | 3.6135 | 525 | 146 | 8.1 | 2919 | 16.5 | 0.341 |
| 27 | 24.877 | 3.5762 | 506 | 153 | 8.5 | 3920 | 22.2 | 0.437 |
| 28 | 25.505 | 3.4895 | 480 | 125 | 6.9 | 808 | 4.6 | 0.110 |
| 29 | 26.968 | 3.3034 | 418 | 82 | 4.5 | 1049 | 5.9 | 0.205 |

### Di-nitrate Compound 2-B

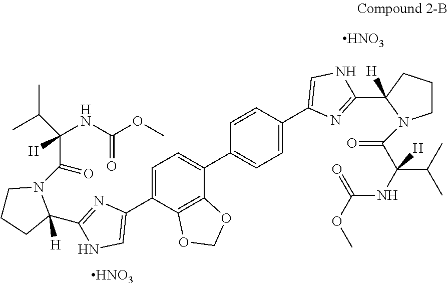

The free base (1 g) was dissolved in 10 mL acetonitrile and the mixture was heated to 70±5° C. 65% HNO₃ (0.25 g, 2 eq.) was added at 70±5° C. with stirring for 0.5 hour and the mixture was cooled to 5±5° C. The resulting solid was filtrated and dried under reduced pressure to afford 0.95 g of crystalline solid Compound 2-B (Yield: 82%). The peaks of the XRPD pattern are shown in Table 3 and the XRPD pattern is shown in FIG. 2.

TABLE 3
Compound 2-B XRPD Pattern Peaks

| Peak No. | 2-Theta | d | BG | Height | Height % | Area | Area % | FWHM |
|---|---|---|---|---|---|---|---|---|
| 1 | 6.836 | 12.9198 | 382 | 139 | 6.6 | 1960 | 6.3 | 0.240 |
| 2 | 8.693 | 10.1638 | 400 | 965 | 46.0 | 7996 | 25.8 | 0.141 |
| 3 | 9.337 | 9.4640 | 428 | 2100 | 100.0 | 17861 | 57.6 | 0.145 |
| 4 | 10.641 | 8.3067 | 391 | 452 | 21.5 | 4649 | 15.0 | 0.175 |
| 5 | 12.426 | 7.1174 | 374 | 154 | 7.3 | 1735 | 5.6 | 0.192 |
| 6 | 14.150 | 6.2541 | 405 | 584 | 27.8 | 7869 | 25.4 | 0.230 |
| 7 | 14.468 | 6.1171 | 426 | 538 | 25.6 | 4287 | 13.8 | 0.136 |
| 8 | 15.193 | 5.8267 | 418 | 2000 | 95.2 | 23781 | 76.7 | 0.203 |
| 9 | 15.512 | 5.7077 | 408 | 523 | 24.9 | 5243 | 16.9 | 0.171 |
| 10 | 19.099 | 4.6430 | 423 | 688 | 32.8 | 7347 | 23.7 | 0.182 |
| 11 | 20.365 | 4.3572 | 502 | 140 | 6.7 | 651 | 2.1 | 0.079 |
| 12 | 21.067 | 4.2135 | 522 | 326 | 15.5 | 9806 | 31.6 | 0.513 |
| 13 | 21.429 | 4.1432 | 558 | 2065 | 98.3 | 30992 | 100.0 | 0.256 |
| 14 | 21.688 | 4.0943 | 586 | 1312 | 62.5 | 23463 | 75.7 | 0.305 |
| 15 | 22.389 | 3.9676 | 550 | 393 | 18.7 | 5129 | 16.5 | 0.222 |
| 16 | 23.495 | 3.7834 | 614 | 211 | 10.0 | 1383 | 4.5 | 0.112 |
| 17 | 24.435 | 3.6399 | 570 | 326 | 15.5 | 8134 | 26.2 | 0.425 |
| 18 | 26.039 | 3.4191 | 482 | 201 | 9.6 | 1448 | 4.7 | 0.123 |
| 19 | 26.438 | 3.3684 | 483 | 164 | 7.8 | 1098 | 3.5 | 0.114 |
| 20 | 27.243 | 3.2707 | 502 | 465 | 22.1 | 6772 | 21.9 | 0.248 |
| 21 | 28.010 | 3.1829 | 496 | 295 | 14.0 | 4702 | 15.2 | 0.272 |

### Di-Hydrobromate Compound 2-C

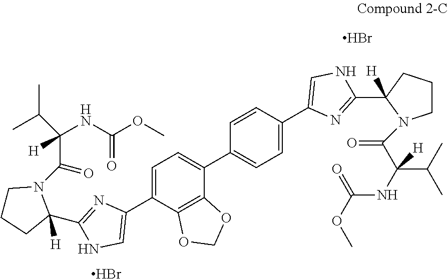 [SMILES: Br ; COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C ; Br]

The free base (0.5 g) was added to i-PrOH (5 mL) and the mixture was heated to 60-70° C. 48% aqueous hydrobromic acid (0.24 g, 2 eq.) was added at this temperature with stirring for 1 hour and then the mixture was cooled to 5±5° C. The resulting solid was filtrated and dried under reduced pressure to afford 0.48 g of crystalline solid Compound 2-C(Yield: 76%). The peaks of the XRPD pattern are shown in Table 4 and the XRPD pattern is shown in FIG. 3.

TABLE 4
Compound 2-C XRPD Pattern Peaks

| Peak No. | 2-Theta | d | BG | Height | Height % | Area | Area % | FWHM |
|---|---|---|---|---|---|---|---|---|
| 1 | 8.513 | 10.3777 | 362 | 248 | 37.0 | 4367 | 22.5 | 0.300 |
| 2 | 9.497 | 9.3048 | 359 | 671 | 100.0 | 11410 | 58.7 | 0.290 |
| 3 | 10.661 | 8.2917 | 325 | 77 | 11.5 | 853 | 4.4 | 0.178 |
| 4 | 13.970 | 6.3341 | 381 | 157 | 23.4 | 2489 | 12.8 | 0.270 |
| 5 | 14.769 | 5.9931 | 415 | 371 | 55.3 | 6866 | 35.3 | 0.315 |
| 6 | 15.372 | 5.7592 | 356 | 534 | 79.6 | 16162 | 83.1 | 0.516 |
| 7 | 16.981 | 5.2172 | 319 | 122 | 18.2 | 2042 | 10.5 | 0.285 |
| 8 | 19.045 | 4.6562 | 373 | 274 | 40.8 | 5056 | 26.0 | 0.315 |
| 9 | 20.804 | 4.2662 | 490 | 135 | 20.1 | 587 | 3.0 | 0.074 |
| 10 | 21.489 | 4.1317 | 442 | 523 | 77.9 | 19441 | 100.0 | 0.634 |
| 11 | 22.029 | 4.0317 | 494 | 304 | 45.3 | 6291 | 32.4 | 0.353 |
| 12 | 23.030 | 3.8587 | 531 | 480 | 71.5 | 7743 | 39.8 | 0.275 |
| 13 | 24.156 | 3.6813 | 561 | 402 | 59.9 | 13366 | 68.8 | 0.567 |
| 14 | 26.237 | 3.3938 | 478 | 164 | 24.4 | 2779 | 14.3 | 0.289 |
| 15 | 27.608 | 3.2283 | 472 | 157 | 23.4 | 2040 | 10.5 | 0.221 |
| 16 | 28.786 | 3.0988 | 458 | 161 | 24.0 | 2697 | 13.9 | 0.286 |
| 17 | 29.805 | 2.9952 | 427 | 85 | 12.7 | 772 | 4.0 | 0.155 |
| 18 | 30.912 | 2.8904 | 365 | 170 | 25.3 | 6516 | 33.5 | 0.653 |

### Example 4. Synthesis of Compound 2 and Compound 2-A

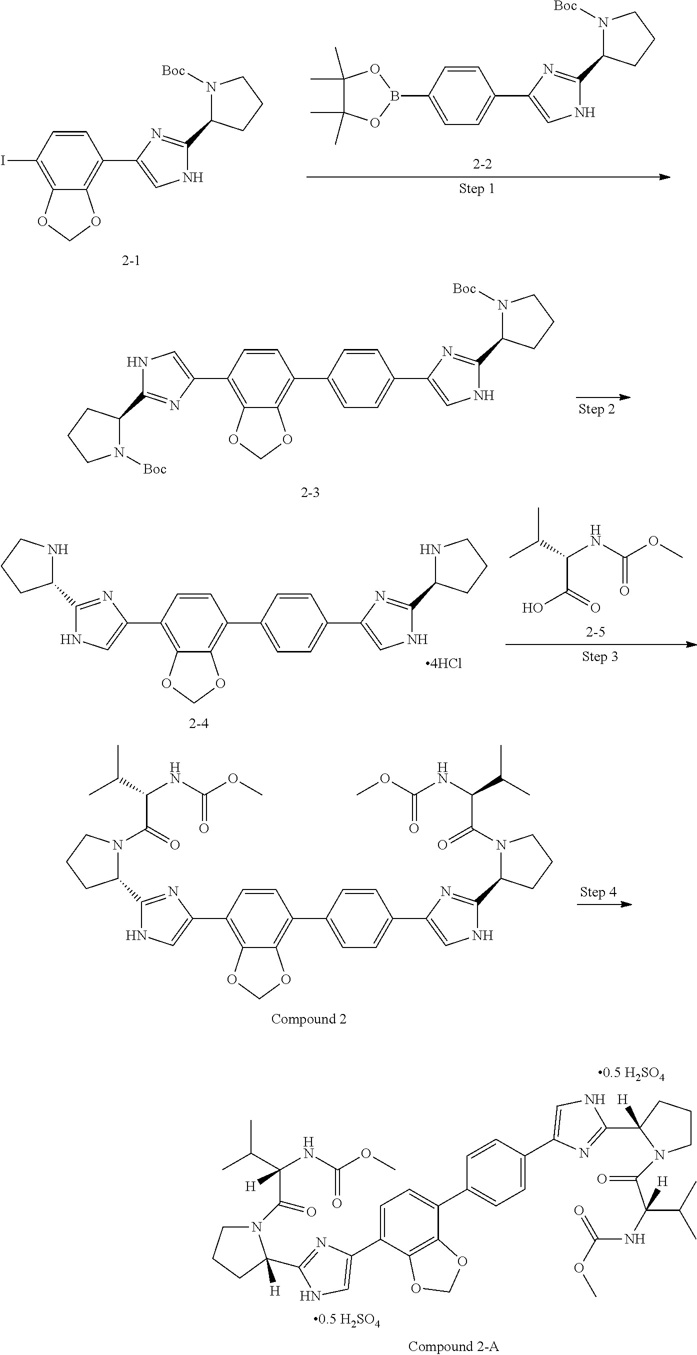

Step 1:

A reactor was charged with compound 2-1 (6 kg) and toluene (46.8 kg) and the mixture was heated to 70±5° C. before activated charcoal (0.6 kg) was added and the mixture was stirred for 60 minutes. The mixture was then filtered and the resulting cake was washed with toluene (5 kg). The filtrate was cooled to 25±5° C. A plastic drum was charged with K₃PO4 (5.4 kg) and portable water (12 kg) (Solution A) and the mixture was stirred. The reactor was then charged with Solution A, 96% ethanol (9.6 kg), compound 2-2 (5.4 kg), and Pd(dppf)Cl2CH₂C₁₂ (0.18 kg) and the reaction mixture was heated to 70±5° C. for 30 hours. N-Acetyl-L-cysteine (0.42 kg) was added at 70±5° C. and the solution was then cooled to 0-5° C. and stirred for 1-2 hours. The reaction was then subjected to centrifugation and the resulting cake was washed with toluene (5 kg). The wet material was added to methanol (24 kg) and the mixture was heated to reflux for 1 hour. The mixture was then cooled to 25±5° C. and stirred for 30 minutes. The mixture was once against subjected to centrifugation and the resulting cake was washed with methanol (5-10 kg). The wet material was dried at 60±5° C. until loss-on-drying (LOD) was not more than 3.0% to afford 7.2 kg of compound 2-3. Yield 120.0% (w/w).

Step 2:

A reactor was charged with portable water (21 kg), isopropanol (5.6 kg), and compound 2-3 (7 kg) and the mixture was heated to 70±5° C. Hydrochloric acid (6.3 kg) was added slowly at 70±5° C. and the reaction stirred for 1-2 hours. Activated charcoal (0.7 kg) was added at 70±5° C. and the reaction was stirred for 60 minutes. The mixture was then filtered and the resulting cake was washed with portable water (5 kg). Isopropanol (82.6 kg) was added at 70±5° C. and the reaction stirred for 1-2 hours. The solution was cooled to 0-5° C. and stirred for 30 minutes. The reaction was then subjected to centrifugation and the resulting cake was washed with isopropanol (5 kg). The wet material was dried at 60±5° C. until loss-on-drying (LOD) was not more than 3.0% to afford 5.35 kg of compound 2-4. Yield 76.4% (w/w).

Step 3: A reactor was charged with DCM (84.27 kg), HOBT (2.92 kg), Moc-L-Val-OH (3.66 kg) and EDCL (3.98 kg) and the resulting solution was stirred. Compound 2-4 (5.3 kg) was added and the mixture was cooled to −20±10° C. The mixture was further cooled to −10° C. and DIPEA (9.54 kg) was added. The reaction was stirred at −20±10° C. for 2-3 hours. The mixture was then heated to 25±5° C. and portable water (15.9 kg) was added. The reaction was stirred for 10-20 minutes. The organic and aqueous layers were separated and portable water (15.9 kg) was added to the organic phase. The temperature was controlled at 25±5° C. before hydrochloric acid (1.59 kg) was added to achieve a pH of approximately 5-6. The organic layer was separated from the aqueous later and portable water (15.9 kg) was added to the organic phase. The mixture was stirred for 10-20 minutes. The resulting phases were again separated and the organic phase was washed with 10% Na₂CO₃ solution (3.18 kg) twice and portable water (44.52 kg) once. The organic phase was concentrated to dryness at a temperature below 60° C. and a vacuum below −0.08 Mpa. Methanol (25.6 kg) was then added and the resulting solution was stirred before H₂SO₄ (1.69 kg) was added slowly at 50±5° C. and the reaction was stirred at 50±5° C. for 1-2 hours. The solution was cooled to 25±5° C. and portable water (10.6 kg) was added before Na₂CO₃ (1.83 kg) was added to achieve a pH of approximately 8-9. The mixture was concentrated to remove methanol at a temperature below 60° C. and a vacuum below −0.08 Mpa. The reaction was charged with ethyl acetate (63.6 kg) and portable water (15.9 kg) and stirred for 30 minutes. The organic and aqueous phases were then separated and the organic phase was washed with portable water (15.9 kg) and concentrated to dryness at a temperature below 60° C. and a vacuum below −0.08 Mpa. Ethyl acetate (28.62 kg) was added and the mixture was stirred to afford Compound 2 ethyl acetate solution. The solution was added slowly into n-hexane (63.07 kg) and the resulting mixture was stirred at 25±5° C. for 1 hour and centrifuged. The resulting cake was washed with a mixture of ethyl acetate (2 kg) and n-hexane (6 kg) and the wet material was dried at 60±5° C. until loss-on-drying (LOD) was not more than 5.0% to afford 6.1 kg of Compound 2. Yield 115.1% (w/w).

Step 4:

A reactor was charged with methanol (14 kg) and Compound 2 (5.8 kg) and the mixture was heated to 35±5° C. Activated charcoal (0.145 kg) was added at 35±5° C. and the mixture stirred for 30 minutes before filtration. The resulting cake was washed with methanol (5 kg). The temperature of the filtrate was brought to 55±5° C. and H₂SO₄ (0.765 kg) was added. The reaction was stirred at 65±5° C. for 2 hours and then cooled to 25±5° C. and stirred for 10 hours. Ethyl acetate (15.7 kg) was added to the reactor and the mixture stirred for 2 hours before centrifugation. The resulting cake was washed with methanol (5 kg) and the wet material was dried at 60±5° C. until loss-on-drying (LOD) was not more than 5.0% to afford 5.6 kg of Compound 2-A. Yield 96.55% (w/w).

Compound 2-A was characterized by ¹HNMR, ¹³CNMR, FI-IR, and mass spectrum in addition to XRPD (FIG. 1A) and differential scanning calorimetry (DSC) (FIG. 1B). The hygroscopicity was measured by placing a sample in a climatic cabinet set at 25±1° C./80±2% RH for 24 hours. The water content was increased from 3.3% to 9.4%.

### Example 5. Stability of Compound 2-A

The stability of Compound 2-A was measured under three different conditions: 1) open container; 2) PE/ALU bag where the PE bag is closed with clips and the ALU bag is sealed by thermos-sealing; and 3) PE/ALU bag with desiccant where the PE bag is closed with plastic clip, the ALU bag is sealed by the thermos-sealing, and 10 g of silica gel is placed between the bags. The open container conditions were conducted with two different batches of Compound 2-A. The results of the stability studies are shown in Table 5A, Table 5B, Table 6, and Table 7. The water content of Compound 2-A slowly increased and the purity was not changed under 25° C./60% RH and 40° C./75% RH.

TABLE 5A
Stability of Batch No. 1 under Open Container Conditions

| | Appearance | Purity (HPLC) | Water (KF) | XRPD |
|---|---|---|---|---|
| Initial | Pale-yellow solid | 98.39% | 3.11% | Crystalline |

25° C./60% RH in stability chambers

| | | | | |
|---|---|---|---|---|
| 7 days | Pale-yellow solid | 98.45% | 6.14% | Consistent |
| | | | | with initial |
| 14 days | Pale-yellow solid | 98.61% | 6.28% | / |
| 28 days | Pale-yellow solid | 98.58% | 5.95% | |

30° C./65% RH in stability chambers

| | | | | |
|---|---|---|---|---|
| 7 days | Pale-yellow solid | 98.40% | 6.73% | Consistent |
| | | | | with initial |
| 14 days | Pale-yellow solid | 98.47% | 6.80% | / |
| 28 days | Pale-yellow solid | 98.59% | 6.49% | |

40° C./75% RH in stability chambers

| | | | | |
|---|---|---|---|---|
| 7 days | Pale-yellow solid | 98.45% | 7.13% | Consistent |
| | | | | with initial |
| 14 days | Pale-yellow solid | 98.51% | 7.67% | / |
| 28 days | Pale-yellow solid | 98.53% | 6.96% | |

TABLE 5B
Stability of Batch No. 2 under Open Container Conditions

| | Appearance | Purity (HPLC) | Water (KF) |
|---|---|---|---|
| Initial | White powder | 99.39% | 4.48% |

25° C./60% RH in stability chambers

| | | | |
|---|---|---|---|
| 7 days | White powder | 99.37% | 6.01% |
| 16 days | White powder | 99.41% | 7.69% |

30° C./65% RH in stability chambers

| | | | |
|---|---|---|---|
| 7 days | White powder | 99.38% | 7.50% |
| 16 days | White powder | 99.41% | 6.98% |

40° C./75% RH in stability chambers

| | | | |
|---|---|---|---|
| 7 days | White powder | 99.38% | 7.96% |
| 16 days | White powder | 99.42% | 7.03% |

TABLE 6
Stability of Batch No. 2 under PE/ALU bag Conditions

| | Appearance | Purity (HPLC) | Water (KF) |
|---|---|---|---|
| Initial | White powder | 99.39% | 4.48% |

25° C./60% RH in stability chambers

| | | | |
|---|---|---|---|
| 7 days | White powder | 99.36% | 5.18% |
| 16 days | White powder | 99.41% | 5.56% |

40° C./75% RH in stability chambers

| | | | |
|---|---|---|---|
| 7 days | White powder | 99.37% | 5.42% |
| 16 days | White powder | 99.42% | 5.91% |

TABLE 7
Stability of Batch No. 2 under PE/ALU with desiccant Conditions

| | Appearance | Purity (HPLC) | Water (KF) |
|---|---|---|---|
| Initial | White powder | 99.39% | 4.48% |

25° C./60% RH in stability chambers

| | | | |
|---|---|---|---|
| 7 days | White powder | 99.38% | 5.46% |
| 16 days | White powder | 99.41% | 5.83% |

40° C./75% RH in stability chambers

| | | | |
|---|---|---|---|
| 7 days | White powder | 99.37% | 5.51% |
| 16 days | White powder | 99.42% | 6.90% |

### Example 6. In Vitro Inhibitory Effects of the Combination of Compound 1-A and Compound 2 on HCV Replicons

The individual EC₅₀ values for Compound 1-A and Compound 2 for each type of HCV replicon (GT1a, GT1b, and GT1b_3a-NS5B) were first determined. Huh7 cells were maintained in DMEM supplemented with 10% FBS, 1% NEAA, 1% L-Glutamine and 1% Penicillin-Streptomycin. The stable HCV GT1a and 1b replicon cells were generated by transfection of Huh7 cells with in vitro transcribed HCV replicon RNA transcripts from replicon DNA constructs and were selected with 250 μg/ml of G418. The GT1b/3a NS5B chimeric replicons were constructed with the GT1b replicon as a backbone. GT1b/3a NS5B replicon RNAs were in vitro transcribed using the replicon plasmid DNAs and used to transiently transfect Huh7 cells by electroporation.

Stock solutions (20 mM) of Compound 1-A and Compound 2 were prepared in 100% DMSO. The final concentration of DMSO in the cell culture medium was 0.5%. Compounds were individually tested for their inhibitory activity in stably-transfected GT1a and GT1b replicons and in transiently-transfected GT1b/3a NS5B chimeric replicons in duplicate at 9 concentrations with a series of 4-fold dilutions starting at 10,000 nM. Replicon cells were seeded (8,000 cells/well for GT1a and 1b; 10,000 cells/well for GT1b/3a NS5B) in 96-well plates containing serially diluted compounds and cultured at 37° C. and 5% CO₂ for 3 days. CellTiter-Fluor was used to detect the fluorescence signal and the raw data (RFU) was used for calculating cell viability using the equation below:

% Viability=(CPD−HPE)(ZPE−HPE)×100

where CPD is a signal from a well containing a test compound, ZPE is the average of signals from DMSO control wells and HPE is the average of signals from medium control wells. Britelite plus was used to detect the luminescent signal and the raw data (RLU) was used for calculating antiviral activity (% inhibition) of the compounds using the equation below:

% Inhibition=(CPD−ZPE)

The data were analyzed using CompuSyn software (ComboSyn, Inc., Paramus, N.J.) to obtain the concentrations of the individual compounds required to achieve 50% cytotoxicity (CC₅₀) and 50% (EC₅₀) and 90% (EC₉₀) antiviral efficacy. These values are shown in Table 8.

TABLE 8
Individual EC₅₀ values of Compound 1-A and Compound 2

Individual EC₅₀ (nM)

| HCV Genotype | Compound 1-A | Compound 2 |
|---|---|---|
| GT1a ¹ | 2.83 | 0.030 |
| GT1b ¹ | 2.69 | 0.014 |
| GT1b_3a-NS5B ² | 1.68 | 0.0029 |

¹ Stable HCV replicons prepared by transfection of Huh7 cells.
² Chimeric HCV replicons containing the GT3a-NS5B gene sequences constructed with the GT1b backbone and prepared in transiently-transfected Huh7 cells.

The effect of the combination of Compound 1-A and Compound 2 on the extent of inhibition of viral replication was determined in the presence of 0.125, 0.25, 1, 2, 4 and 8 times the ratio of the individual EC₅₀ values for each HCV genotype (Lowe Additivity Model) using the methods described above. Test concentrations of the compounds were 0.125, 0.25, 0.5, 1, 2, 4 and 8× the EC₅₀ values. The test concentrations of the compounds are shown in Table 9. The final concentration of DMSO in cell culture medium was 0.5%.

TABLE 9
The Final Concentrations of Compounds tested in the Combination Experiment

| Replicon | Compd | Compound concentrations (nM) | | | | | | | |
|---|---|---|---|---|---|---|---|---|---|
| GT1a wild | 1-A | 22.65 | 11.32 | 5.662 | 2.831 | 1.416 | 0.708 | 0.354 | 0 |
| type | 2 | 0.242 | 0.121 | 0.0605 | 0.0303 | 0.0151 | 0.0076 | 0.0038 | 0 |
| GT1b wild | 1-A | 21.50 | 10.75 | 5.374 | 2.687 | 1.344 | 0.672 | 0.336 | 0 |
| type | 2 | 0.111 | 0.056 | 0.028 | 0.014 | 0.0069 | 0.0035 | 0.0017 | 0 |
| GT1b/3a | 1-A | 13.43 | 6.716 | 3.358 | 1.679 | 0.840 | 0.420 | 0.210 | 0 |
| NS5A | 2 | 0.275 | 0.138 | 0.069 | 0.034 | 0.017 | 0.0086 | 0.0043 | 0 |
| GT1b/3a | 1-A | 14.98 | 7.488 | 3.744 | 1.872 | 0.936 | 0.468 | 0.234 | 0 |
| NS5B | 2 | 0.023 | 0.011 | 0.0057 | 0.0029 | 0.0014 | 0.00071 | 0.00036 | 0 |

CompuSyn was also used to create plots (isobolograms) of the 90% inhibition levels expected for each genotype assuming a strictly additive antiviral effect of the combination of the two compounds, and to obtain and plot the values on the isobolograms that represents the concentrations of each individual compound required to achieve a 90% antiviral effect when combined at the ratio of their individual EC₅₀ values for each genotype (combination index at 90% inhibition; CI₉₀). As per the Lowe Additivity Model, CI values equal to 1 (lying on the isobologram), greater than 1 (lying above the isobologram) and less than 1 (lying below the isobologram) represent effects of the combination of two compounds that are additive, antagonistic and synergistic, respectively.

The CI was less than 1 for all genotypes tested, indicating a synergistic combination effect. The CI for three genotypes (GT1a, GT1b, and GT1b_3a-NS5B) is shown in Table 10.

TABLE 10
Combination Index for Combination
of Compound 1-A and Compound 2

| | Lowe Additivity Model | |
|---|---|---|
| | Combination | Combination |
| HCV Genotype | Index (EC₉₀) | Effect |
| GT1a | 0.659 | Synergistic |
| GT1b | 0.746 | Synergistic |
| GT1b_3a-NS5B | 0.817 | Synergistic |

The isobologram for GT1a, GT1b, and GT1b_3a-NS5B are shown as FIG. 4A, FIG. 4B, and FIG. 4C, respectively. The x-axis represents the concentration of Compound 2 required to achieve 90% inhibition and the y-axis represents the concentration of Compound 1-A required to achieve 90% inhibition. The dose at which Compound 1-A alone achieves 90% inhibition is plotted and the dose at which Compound 2 alone achieves 90% inhibition is plotted. These two points are then connected to form a line of additivity. The concentration of Compound 1-A and Compound 2 used in combination to provide the same effect (i.e., 90% inhibition) is also plotted and represented by the star (*). In each of these isobolograms, the star is below the line of additivity, again indicating a synergistic effect.

In Example 6, Huh7 cells were transiently transfected with replicon RNA by electroporation and seeded at a density of 10,000 cells/well in 96-well plates. HCV GT1a and GT1b replicons stable cells were seeded at a density of 8,000 cells/well in 96-well plates. Cells were cultured and treated with the compounds at 37° C. and 5% CO₂ for 3 days. Cell viability was assessed with CellTiter-Fluor in accordance with the protocol provided by the supplier. The CellTiter-Fluor reagent was added to wells and incubated at 5% CO₂ and 37° C. for 1 hour. Fluorimetric signal was measured with an Envision. The raw fluorimetric signal data (RFU) was used to calculate the cell viability using the equation above.

The antiviral activity of the compounds was determined by monitoring activity of replicon reporter firefly luciferase using Britelite plus in accordance with the protocol provided by the supplier. The combination indices were calculated using the MacSynergy™ II software (Prichard and Shipman, 1990). A positive combination index value indicates synergism, and a negative combination index value indicates antagonism.

### Example 7. Non-Limiting Examples of Solid Dosage Formulations

Representative non-limiting batch formulas for Compound 1-A and 2-A tablets (60 mg and 100 mg) are presented in Table 11 and Table 12. The tablets are produced from a common blend using a direct compression process. The active pharmaceutical ingredient (API) is adjusted based on the as-is assay, with the adjustment made in the percentage of microcrystalline cellulose.

Compound 1-A and excipients (microcrystalline cellulose, lactose monohydrate, and croscarmellose sodium) are screened, placed into a V-blender (PK Blendmaster, 0.5 L bowl) and mixed for 5 minutes at 25 rpm. Magnesium stearate is then screened and added followed by Compound 2-A. The common blend is divided for use in producing 60 mg and 100 mg tablets. The lubricated blend is then compressed at a speed of 10 tablets/minutes using a single punch research tablet press (Korsch XP1) and a gravity powder feeder. The 60 tablets are produced using round standard concave 6 mm tooling and 3.5 kN forces. The 100 mg tablets are produced using 8 mm round standard concave tooling and 3.9-4.2 kN forces.

TABLE 11
Non-limiting Examples of Formulations of 60 mg Tablets

| | Example 1 | Example 2 |
|---|---|---|
| Raw Material | mg/unit | mg/unit |
| Compound 1-A | 600^{a} | 600^{a} |
| Compound 2-A | 67^{b} | 67^{b} |
| Silicified Microcrystalline Cellulose, HD 90, NF | 357 | 293 |
| Mannitol EP, USP | 162.0 | 162.0 |
| Croscarmellose Sodium, USP/NF, EP | 60.0 | 60.0 |
| Magnesium Stearate, USP/NF, BP, EP JP | 18.0 | 18.0 |
| Total | 1264 | 1200 |

^{a}equivalent to 550 mg of Compound 1
^{b}equivalent to 60 mg of Compound 2

TABLE 12
Non-limiting Examples of Formulations of 100 mg Tablets

| | Example 1 | Example 2 |
|---|---|---|
| Raw Material | mg/unit | mg/unit |
| Compound 1-A | 600^{a} | 600^{a} |
| Compound 2-A | 113^{b} | 113^{b} |
| Silicified Microcrystalline Cellulose, HD 90, NF | 357 | 247 |
| Mannitol EP, USP | 162.0 | 162.0 |
| Croscarmellose Sodium, USP/NF, EP | 60.0 | 60.0 |
| Magnesium Stearate, USP/NF, BP, EP JP | 18.0 | 18.0 |
| Total | 1310 | 1200 |

^{a}equivalent to 550 mg of Compound 1
^{b}equivalent to 100 mg of Compound 2

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

What is claimed is:

1. A pharmaceutical dosage form comprising an effective amount of Compound 1 or a pharmaceutically acceptable salt thereof and an effective amount of Compound 2 or a pharmaceutically acceptable salt thereof:

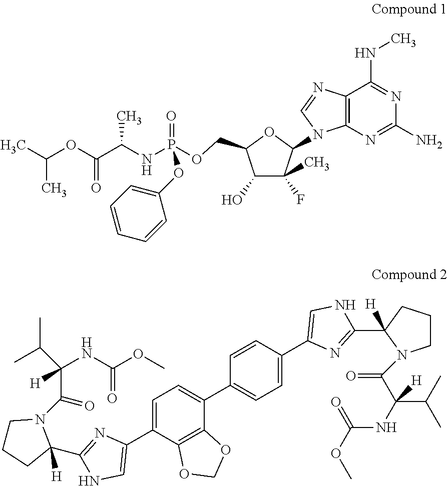 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F ; COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

2. The pharmaceutical dosage form of claim 1, wherein Compound 1 is Compound 1-A:

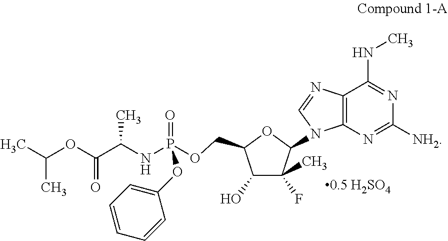 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

3. The pharmaceutical dosage form of claim 1, wherein Compound 2 is Compound 2-A:

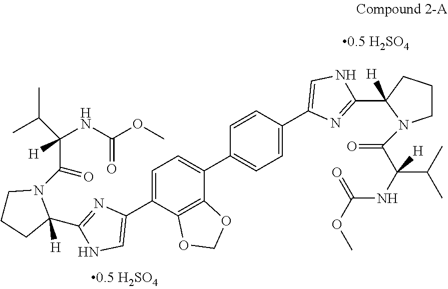 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

4. The pharmaceutical dosage form of claim 1, wherein the effective amount of Compound 1 or a pharmaceutically acceptable salt thereof and the effective amount of Compound 2 or a pharmaceutically acceptable salt thereof are administered in a single fixed-dosage form.

5. The pharmaceutical dosage form of claim 1, wherein the dosage form is suitable for oral delivery.

6. The pharmaceutical dosage form of claim 5, wherein the dosage form is a tablet.

7. The pharmaceutical dosage form of claim 5, wherein the dosage form is a capsule.

8. The pharmaceutical dosage form of claim 1, wherein the composition is in a dosage form suitable for delivery selected from parenteral, intravenous, intramuscular, topical, transdermal, buccal, subcutaneous and suppository.

9. Compound 2-A of the formula:

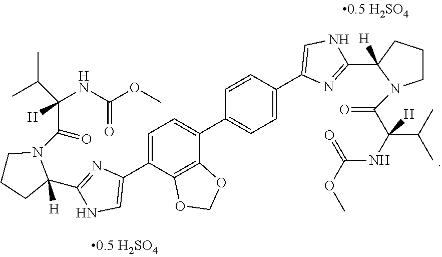 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]

10. The Compound 2-A of claim 9, in solid form.

11. The Compound 2-A of claim 10, in a substantially crystalline form.

12. An isolated crystalline form of Compound 2-A: 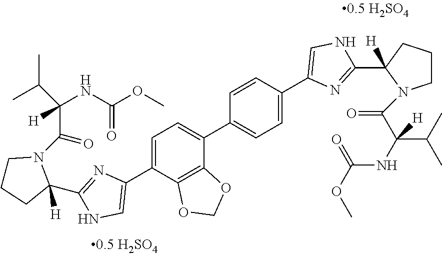 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)c4c3OCO4)cc2)c[nH]1)C(C)C]
characterized by an X-ray diffraction (XRPD) pattern comprising at least five 2theta values selected from 7.3±0.2°, 7.9±0.2°, 12.0±0.2°, 12.2±0.2°, 14.7±0.2°, 15.8±0.2°, 16.1±0.2°, 16.5±0.2°, 18.2±0.2°, and 22.7±0.2°.

13. The isolated crystalline form of Compound 2-A of claim 12, characterized by an X-ray diffraction (XRPD) pattern comprising at least six 2theta values selected from 7.3±0.2°, 7.9±0.20, 12.0±0.2°, 12.2±0.2°, 14.7±0.2°, 15.8±0.2°, 16.1±0.2°, 16.5±0.2°, 18.2±0.2°, and 22.7±0.2°.

14. The isolated crystalline form of Compound 2-A of claim 12, characterized by an X-ray diffraction (XRPD) pattern comprising at least seven 2theta values selected from 7.3±0.2°, 7.9±0.20, 12.0±0.2°, 12.2±0.2°, 14.7±0.2°, 15.8±0.2°, 16.1±0.2°, 16.5±0.2°, 18.2±0.2°, and 22.7±0.2°.

15. The isolated crystalline form of Compound 2-A of claim 12, wherein the X-ray diffraction (XRPD) pattern comprises 2theta values selected from 7.3±0.2°, 7.9±0.2°, 12.0±0.2°, 12.2±0.2°, 14.7±0.2°, 15.8±0.2°, 16.1±0.2°, 16.5±0.2°, 18.2±0.2°, and 22.7±0.2°.

16. The isolated crystalline form of Compound 2-A of claim 12, wherein the X-ray diffraction (XRPD) pattern comprises at least the 2theta value of 7.3±0.2°.

17. The isolated crystalline form of Compound 2-A of claim 12, wherein the X-ray diffraction (XRPD) pattern comprises at least the 2theta value of 18.2±0.2°.

18. The isolated crystalline form of Compound 2-A of claim 12, wherein the X-ray diffraction (XRPD) pattern comprises at least the 2theta value of 14.7±0.2°.

19. The pharmaceutical dosage form of claim 3, wherein Compound 2-A is an isolated crystalline form characterized by an X-ray diffraction (XRPD) pattern comprising 2theta values selected from 7.3±0.2°, 7.9±0.2°, 12.0±0.2°, 12.2±0.2°, 14.7±0.2°, 15.8±0.2°, 16.1±0.2°, 16.5±0.2°, 18.2±0.2°, and 22.7±0.2°.

20. The pharmaceutical dosage form of claim 3, wherein Compound 2-A is an isolated crystalline form characterized by an X-ray diffraction (XRPD) pattern comprising at least five 2theta values selected from 7.3±0.2°, 7.9±0.2°, 12.0±0.2°, 12.2±0.2°, 14.7±0.2°, 15.8±0.2°, 16.1±0.2°, 16.5±0.2°, 18.2±0.2°, and 22.7±0.2°.

21. A kit that comprises a dosage form comprising Compound 1 or a pharmaceutically acceptable salt thereof and a dosage form comprising Compound 2 or a pharmaceutically acceptable salt thereof.

22. The kit of claim 21, wherein Compound 1 is Compound 1-A.

23. The kit of claim 21, wherein Compound 2 is Compound 2-A.

24. A method of treating a hepatitis C infection in a patient in need thereof comprising administering an effective amount of Compound 1 or a pharmaceutically acceptable salt thereof in combination with an effective amount of Compound 2 or a pharmaceutically acceptable salt thereof.

25. The method of claim 24, which provides overlapping AUC pharmacokinetics of Compound 1 or a pharmaceutically acceptable salt thereof and Compound 2 or a pharmaceutically acceptable salt thereof.

26. The method of claim 24, wherein Compound 1 or a pharmaceutically acceptable salt thereof and Compound 2 or a pharmaceutically acceptable salt thereof provide a synergistic effect.

27. The method of claim 24, wherein Compound 1 or a pharmaceutically acceptable salt thereof and Compound 2 or a pharmaceutically acceptable salt thereof are administered in a single fixed-dosage form.

28. The method of claim 24, wherein Compound 1 or a pharmaceutically acceptable salt thereof and Compound 2 or a pharmaceutically acceptable salt thereof are administered in separate dosage forms.

29. The method of claim 24, wherein Compound 1 is Compound 1-A.

30. The method of claim 24, wherein Compound 2 is Compound 2-A.

31. The method of claim 30, wherein Compound 2-A is an isolated crystalline form characterized by an X-ray diffraction (XRPD) pattern comprising 2theta values selected from 7.3±0.2°, 7.9±0.2°, 12.0±0.2°, 12.2±0.2°, 14.7±0.2°, 15.8±0.2°, 16.1±0.2°, 16.5±0.2°, 18.2±0.2°, and 22.7±0.2°.

32. The method of claim 24, wherein the treatment period is 24 weeks or less.

33. The method of claim 24, wherein the treatment period is 12 weeks or less.

34. The method of claim 24, wherein the treatment period is 8 weeks or less.

35. The method of claim 24, wherein the patient is cirrhotic.

36. The method of claim 24, wherein the patient is non-cirrhotic.

37. The method of claim 24, wherein the HCV comprises genotype 1.

38. The method of claim 37, wherein the HCV comprises genotype 1a.

39. The method of claim 37, wherein the HCV comprises genotype 1b.

40. The method of claim 24, wherein the HCV comprises genotype 2.

41. The method of claim 24, wherein the HCV comprises genotype 3.

42. The method of claim 41, wherein the HCV comprises genotype 3a.

43. The method of claim 41, wherein the HCV comprises genotype 3b.

44. The method of claim 24, wherein the HCV comprises genotype 4.

45. The method of claim 24, wherein the HCV comprises genotype 5.

46. The method of claim 24, wherein the HCV comprises genotype 6.

47. The method of claim 24, wherein the composition exhibits pan-genotypic efficacy.

48. The method of claim 24, wherein the composition is administered once per day during the period of administration.

49. The method of claim 24, wherein Compound 1 is administered in an amount that is about 550 mg per day.

50. The method of claim 24, wherein Compound 1-A is administered in an amount that is about 600 mg per day.

51. The method of claim 24, wherein Compound 2 is administered in an amount that that is about 60 mg per day.

52. The method of claim 24, wherein Compound 2-A is administered in an amount that that is about 67 mg per day.
